SEQUENCE LISTING (11 sequences)

SEQ ID NO: 1 - protein, 560 aa, Drosophila melanogaster
MASPANPSSNHKMNGNGKIYCEGNLLHTIQTAVPKLFADSKTFVDMKLNNSPDKTLEDFNAMMEAKNQTPSSEDLKQFVDKYFSAPGTELEKWTPTDWKENPSFLDLISDPDLKQWGVELNSIWKDLGRKMKDEVSKNPEYYSIIPVPNPVIVPGGRFIEFYYWDSYWIIRGLLYSQMFDTARGMIENFFSIVNRFGFIPNGGRVYYHGRSQPPLLTGMVKSYVDFTNDDKFAIDALDTLEHEFEFFVNNHNVTVKNHSLCVYRDSSSGPRPESYREDVETGEEFPTDEAKELHYSELKAGAESGMDFSSRWFISPTGTNDGNRSALSTTSIVPVDLNAYLYWNAKLIAEFHSKAGNTKKVTEYETKAEKLLLGIQEVLWNEEAGVWLDYDMINQKPRDYYTPTNLSPLWVKAFNISESEKISASVMAYIERNKLDSFPGGVPNTLSYTGEQWDAPNVWAPMQYILVEGLNNLNTPEAKNMSLKWATRWVKTNFAAFSKDRHMYEKYNADEFGVGGGGGEYEVQTGFGWSNGVIIEWLSKHGRDISIGSGCGCLAGEKRQ

SEQ ID NO: 2 - protein, 515 aa, Drosophila melanogaster
MKLNNSPDKTLEDFNAMMEAKNQTPSSEDLKQFVDKYFSAPGTELEKWTPTDWKENPSFLDLISDPDLKQWGVELNSIWKDLGRKMKDEVSKNPEYYSIIPVPNPVIVPGGRFIEFYYWDSYWIIRGLLYSQMFDTARGMIENFFSIVNRFGFIPNGGRVYYHGRSQPPLLTGMVKSYVDFTNDDKFAIDALDTLEHEFEFFVNNHNVTVKNHSLCVYRDSSSGPRPESYREDVETGEEFPTDEAKELHYSELKAGAESGMDFSSRWFISPTGTNDGNRSALSTTSIVPVDLNAYLYWNAKLIAEFHSKAGNTKKVTEYETKAEKLLLGIQEVLWNEEAGVWLDYDMINQKPRDYYTPTNLSPLWVKAFNISESEKISASVMAYIERNKLDSFPGGVPNTLSYTGEQWDAPNVWAPMQYILVEGLNNLNTPEAKNMSLKWATRWVKTNFAAFSKDRHMYEKYNADEFGVGGGGGEYEVQTGFGWSNGVIIEWLSKHGRDISIGSGCGCLAGEKRQ

SEQ ID NO: 3 - protein, 596 aa, Drosophila melanogaster
MFKLPTISLLLVSWSCLVALSQAKTYSLPDLTTDYNNAIPVDEEEAQDPFASCKIYCEGNLLHTIQTAVPKLFADSKTFVDMKLNNSPDKTLEDFNAMMEAKNQTPSSEDLKQFVDKYFSAPGTELEKWTPTDWKENPSFLDLISDPDLKQWGVELNSIWKDLGRKMKDEVSKNPEYYSIIPVPNPVIVPGGRFIEFYYWDSYWIIRGLLYSQMFDTARGMIENFFSIVNRFGFIPNGGRVYYHGRSQPPLLTGMVKSYVDFTNDDKFAIDALDTLEHEFEFFVNNHNVTVKNHSLCVYRDSSSGPRPESYREDVETGEEFPTDEAKELHYSELKAGAESGMDFSSRWFISPTGTNDGNRSALSTTSIVPVDLNAYLYWNAKLIAEFHSKAGNTKKVTEYETKAEKLLLGIQEVLWNEEAGVWLDYDMINQKPRDYYTPTNLSPLWVKAFNISESEKISASVMAYIERNKLDSFPGGVPNTLSYTGEQWDAPNVWAPMQYILVEGLNNLNTPEAKNMSLKWATRWVKTNFAAFSKDRHMYEKYNADEFGVGGGGGEYEVQTGFGWSNGVIIEWLSKHGRDISIGSGCGCLAGEKRQ

SEQ ID NO: 4 - DNA, 1683 bp, Drosophila melanogaster
  source 1..1683: mol_type = other DNA
atggcctctccagcgaatccatcgagcaatcacaaaatgaacggaaatggtaaaatctactgcgagggcaatctgctgcacaccatccaaacggcagtgcccaaactatttgcggattcgaaaacgtttgtggacatgaagctgaacaattcgcccgacaagaccctcgaggactttaatgccatgatggaggccaagaatcagacgccaagcagtgaggatctcaagcagtttgtcgataagtacttcagtgcaccgggcaccgagcttgagaaatggacgcccaccgactggaaggagaatcccagtttcctcgacctgatctccgacccagatctgaagcaatggggcgtcgagctgaatagcatttggaaggacttgggacgcaaaatgaaggacgaggtgtcaaagaatcccgaatactactcaatcattcccgtgccaaatccagtgatcgtgcccggcggtcgtttcattgagttctactactgggactcctactggatcatccgtggacttctctacagccagatgtttgacaccgcgcgcggcatgattgagaacttcttttccattgtcaatcggttcggttttattccaaacggcggtcgagtctactaccacggtcgctcccagccgccacttctaaccggtatggtcaagtcgtacgtggacttcaccaacgatgacaagttcgccatcgatgccctggacacgctggagcacgagttcgagttctttgtgaacaaccacaatgtcacggtgaagaatcacagcctgtgtgtgtaccgcgattcgtcgtccggaccgcgaccagaatcctaccgagaggatgtggagaccggcgaggagttccccacggatgaggccaaggaactgcattacagtgaactcaaggcaggcgccgaatcgggcatggactttagctcgcgctggttcatctcaccgactggaaccaatgatggcaaccggagcgctctgagcaccacctccattgtgcccgtcgacctgaatgcctatctctactggaacgccaagttgattgccgagttccattccaaagcgggcaacaccaaaaaggtcaccgaatacgagaccaaggccgagaaactccttctgggtatccaagaagttttgtggaacgaggaggccggtgtctggttggactacgatatgattaaccagaagcctcgcgattactacacgcccaccaatctatctccactgtgggtgaaggccttcaacatttcggagtccgaaaagatatcggcttcggttatggcctacattgagaggaacaagctggacagcttccctggcggagttcccaacacgctgagctacaccggagaacagtgggatgcccccaatgtgtgggcaccgatgcagtacatcctggtcgagggcctaaacaacctgaacactcccgaggccaagaatatgtcactgaagtgggccaccaggtgggtgaagacaaactttgcggcgtttagcaaggacaggcacatgtacgagaagtacaacgccgatgagttcggagttggaggcggcggtggcgagtacgaggtacagactggattcggttggtccaacggtgtgatcatcgagtggctgagcaagcacgggcgcgacatttccattggatccggttgtggctgcctagcaggtgaaaagcggcagtag

SEQ ID NO: 5 - protein, 857 aa, Drosophila melanogaster
MSGRDNRGAGGGGGGHQPLSNAMGKLKEKLTRVGDELGYHRVESNLSTSNTATSLDTILPEDPFLFPQVSPQRHPQNTVRTQRLLEDEPPLSFRPLLEDDDINEPPTQQQQRTPLRASGSLELTPLPPPPTSLEIREHRDRQQRGAQGDELQRSKQSLKGSRVSFERRDTGNSNTNSNKAAESSDEDSFEEKRTGFQQQKATSVDHKGILKDLKHILANDNRRQFQAKKHVSLDVKGTRFLQDLLKESSSEEEFHKTRREFQGRKHQSLDPRVTFKLDKVLQGSSTDSDEEGEDAEHKRLIHRPKDITKPVIIDLKDLESESDEDFLTSRQHFQQQRSISTDSRKSRRLYEMDEMDNKRGENIRHAVPFVRQITEDGKPKLEVYRPTTNPIYIWTQVLAALSVSLGSLVVGFVSAYTSPALVSMTDRNITSFEVTQDAGSWVGGIMPLAGLAGGIAGGPLIEYLGRRNTILATAVPFIVSSLLIACAVNVAMVLCGRFLAGFCVGIASLSLPVYLGETVQPEVRGTLGLLPTAFGNIGILLCFVAGSFMNWSMLAFLGAALPVPFLILMFLIPETPRWFVGRGLEERARKALKWLRGKEADVEPELKGLMRSQADADRQASRNTMLELLKLNNLKPLSISLGLMFFQQFSGINAVIFYTVQIFKDAGSTIDGNLCTIIVGIVNFLATFIGIVLIDRAGRKILLYVSDIAMVLTLFVLGGFFYCKTYGPDVSHLGWLPLTCFVIYILGFSLGFGPIPWLMMGEILPAKIRGSAASVATAFNWFCTFVVTKTFQDLTVAMGAHGAFWLFGAICFVGLFFVIIYVPETQGKTLEDIERKMMGRVRRMSSVANIKPLSFNM

SEQ ID NO: 6 - protein, 489 aa, Drosophila melanogaster
MKILMRADTHVSFSVPVEEPKAICTFSQVLAALSVSLGSLVVGFVSAYTSPALVSMTDRNITSFEVTQDAGSWVGGIMPLAGLAGGIAGGPLIEYLGRRNTILATAVPFIVSSLLIACAVNVAMVLCGRFLAGFCVGIASLSLPVYLGETVQPEVRGTLGLLPTAFGNIGILLCFVAGSFMNWSMLAFLGAALPVPFLILMFLIPETPRWFVGRGLEERARKALKWLRGKEADVEPELKGLMRSQADADRQASRNTMLELLKLNNLKPLSISLGLMFFQQFSGINAVIFYTVQIFKDAGSTIDGNLCTIIVGIVNFLATFIGIVLIDRAGRKILLYVSDIAMVLTLFVLGGFFYCKTYGPDVSHLGWLPLTCFVIYILGFSLGFGPIPWLMMGEILPAKIRGSAASVATAFNWFCTFVVTKTFQDLTVAMGAHGAFWLFGAICFVGLFFVIIYVPETQGKTLEDIERKMMGRVRRMSSVANIKPLSFNM

SEQ ID NO: 7 - DNA, 2574 bp, Drosophila melanogaster
  source 1..2574: mol_type = other DNA
atgagtggacgcgacaacagaggagccggcggcggaggcggcggtcatcaaccgctgagcaacgccatgggcaagctcaaggaaaaactaacccgagtaggtgacgagctgggctatcatcgtgtggagagcaatttgtccacatcgaacacagcgaccagtttggacacaattctgccagaggatccgttcctattcccccaagtgtcaccccaaaggcatccacaaaatacagtaaggacacagcgtttgctggaggacgagccaccgctatcgtttcgcccgcttttggaggacgacgacatcaacgagccacctacacagcagcaacagaggactcccctgcgcgccagcggtagtttggagttgactcccttgccaccgccgcccacatcattggagatacgtgagcatcgcgatcgccagcagagaggtgcccagggtgacgagctgcagcgcagcaagcagagcttgaaaggatcacgcgtgtccttcgagcgacgggataccggaaacagcaataccaacagtaacaaagcagcggagagcagcgacgaggacagtttcgaggagaagcgaactggtttccagcaacagaaggcaaccagcgtcgatcacaagggcatcttgaaggacctgaagcacattttggccaacgacaatcgccggcagtttcaggccaagaagcacgtatccctggacgttaagggcacacgcttcctgcaggatctactgaaggaatcgtcatcggaagaggagttccacaagacacggcgggagttccagggtcgcaagcaccagagtttggatccacgtgtcacattcaagctcgacaaagtcctccagggatcgagtaccgacagtgacgaggagggcgaagatgccgagcacaaacgcctgatccatcgacccaaggacattaccaagccggtgatcatcgatcttaaggatctggaaagcgagagcgacgaggactttctcacatcccgtcagcacttccaacagcagcgttccatcagcacagattcccgcaagagccggcgactctacgagatggacgagatggacaacaaacgcggcgagaacatccgtcatgcggtgcccttcgtgcgccagataaccgaggatggcaagcccaagctggaggtctaccgtcccacaacgaatcccatatacatttggacacaggtcttggctgctctgagtgtctcgctgggctccctagtggtcggatttgtcagcgcttacacgtcgcctgctcttgtgtcgatgaccgatcggaatatcacctcgtttgaggtcacccaagatgctggttcttgggttggtggcatcatgccgctggctggattggcaggtggcatagccggaggacccttgatcgaatatctgggcaggcgcaacaccatcctggccactgcggtgcccttcattgtctcatcactactgattgcctgtgctgtgaatgtggctatggttctgtgcggacgattcctggctggattctgcgttggcattgcctctttatccctgcccgtctacctgggtgaaactgtgcagcctgaggtgcgaggcactttgggtttgctgcccacggcctttggtaacataggcatcctgctctgcttcgtggcgggatcctttatgaattggtcgatgctggccttcttgggagctgctctgcccgttcccttcctgattttaatgttccttattccggaaactccgcgctggtttgtgggccgtggtctggaggagcgcgccaggaaggctttgaaatggctgcgtggcaaggaggctgatgtggagcccgaactgaagggtttgatgcgatcccaggccgatgccgatcgtcaggcatcgcgaaacaccatgttggagctgcttaagctcaacaatctgaagccactatcaatttccttgggtctgatgttcttccaacagttcagcggtatcaacgcggttatcttctacacggtccagatcttcaaggatgcgggatccaccattgatggcaatctctgcacgatcattgtgggaattgttaactttttggctacgtttataggcatcgttctgattgatcgggcgggcagaaagattcttctatatgtctccgacatcgccatggttctgaccctgttcgttctgggcggcttcttctactgcaagacgtacggtccggatgtgtcccatttgggttggttgccactcacttgcttcgtcatctacattctgggattctccctgggcttcggacccattccctggctgatgatgggtgagattctgccggccaaaattcgcggatcggctgcctcggtggccacggctttcaactggttctgcacgtttgtggtgaccaagaccttccaggacctcacggttgctatgggcgcgcatggagcgttctggctattcggcgccatctgcttcgtgggtctgttctttgtgataatctacgtgcccgaaacgcagggcaagacgctggaggacatcgagcggaagatgatgggccgtgtgcgccgcatgtcctcggtggccaatatcaagccattgtccttcaacatgtaa

SEQ ID NO: 8 - DNA, 1470 bp, Drosophila melanogaster
  source 1..1470: mol_type = other DNA
atgaagatcctgatgcgagcggacacccatgtgtccttctccgtgcccgtcgaggagcccaaggccatttgtaccttctcccaggtcttggctgctctgagtgtctcgctgggctccctagtggtcggatttgtcagcgcttacacgtcgcctgctcttgtgtcgatgaccgatcggaatatcacctcgtttgaggtcacccaagatgctggttcttgggttggtggcatcatgccgctggctggattggcaggtggcatagccggaggacccttgatcgaatatctgggcaggcgcaacaccatcctggccactgcggtgcccttcattgtctcatcactactgattgcctgtgctgtgaatgtggctatggttctgtgcggacgattcctggctggattctgcgttggcattgcctctttatccctgcccgtctacctgggtgaaactgtgcagcctgaggtgcgaggcactttgggtttgctgcccacggcctttggtaacataggcatcctgctctgcttcgtggcgggatcctttatgaattggtcgatgctggccttcttgggagctgctctgcccgttcccttcctgattttaatgttccttattccggaaactccgcgctggtttgtgggccgtggtctggaggagcgcgccaggaaggctttgaaatggctgcgtggcaaggaggctgatgtggagcccgaactgaagggtttgatgcgatcccaggccgatgccgatcgtcaggcatcgcgaaacaccatgttggagctgcttaagctcaacaatctgaagccactatcaatttccttgggtctgatgttcttccaacagttcagcggtatcaacgcggttatcttctacacggtccagatcttcaaggatgcgggatccaccattgatggcaatctctgcacgatcattgtgggaattgttaactttttggctacgtttataggcatcgttctgattgatcgggcgggcagaaagattcttctatatgtctccgacatcgccatggttctgaccctgttcgttctgggcggcttcttctactgcaagacgtacggtccggatgtgtcccatttgggttggttgccactcacttgcttcgtcatctacattctgggattctccctgggcttcggacccattccctggctgatgatgggtgagattctgccggccaaaattcgcggatcggctgcctcggtggccacggctttcaactggttctgcacgtttgtggtgaccaagaccttccaggacctcacggttgctatgggcgcgcatggagcgttctggctattcggcgccatctgcttcgtgggtctgttctttgtgataatctacgtgcccgaaacgcagggcaagacgctggaggacatcgagcggaagatgatgggccgtgtgcgccgcatgtcctcggtggccaatatcaagccattgtccttcaacatgtaa

SEQ ID NO: 9 - DNA, 62 bp, synthetic construct
  misc_feature 1..62: note = Synthetis
  source 1..62: mol_type = other DNA
gcgaagcttgccaccatgtacccatacgatgttccagattacgctgcctctccagcgaatcc

SEQ ID NO: 10 - DNA, 32 bp, synthetic construct
  misc_feature 1..32: note = Synthetic
  source 1..32: mol_type = other DNA
cagaattcctactgccgcttttcacctgctag

SEQ ID NO: 11 - protein, 9 aa, synthetic construct
  REGION 1..9: note = Synthetic
KTWGQYWQV

(19) United States
(12) Patent Application Publication
(10) Pub. No.: US 2026/0021180 A1
(43) Pub. Date: Jan. 22, 2026

(54) IMPROVING ADOPTIVE CELL TRANSFER THERAPY (ACT) TREATMENT

(71) Applicant: YISSUM RESEARCH DEVELOPMENT COMPANY OF THE HEBREW UNIVERSITY OF JERUSALEM LTD., Jerusalem (IL)

(72) Inventors: Michael BERGER, Jerusalem (IL); Amijai Oriel SARAGOVI, Jerusalem (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 18/993,479

(22) PCT Filed: Jul. 11, 2023
(86) PCT No.: PCT/IL2023/050721

§ 371 Date: Jan. 10, 2025

Related U.S. Application Data
(60) Provisional application No. 63/388,161, filed on Jul. 11, 2022.

(51) Int. Cl. A61K 40/11; A61K 31/7016; A61K 35/15; A61K 35/17; A61K 40/15; A61K 40/17; A61K 40/19; A61K 40/31; A61K 40/42; A61P 35/00; C12N 5/0783; C12N 5/10; C12N 9/24

(52) U.S. Cl. CPC A61K 40/11; A61K 31/7016; A61K 35/15; A61K 35/17; A61K 40/15; A61K 40/17; A61K 40/19; A61K 40/31; A61K 40/42; A61P 35/00; C12N 5/0636; C12N 5/10; C12N 9/2402; C12Y 302/01028; C12N 2510/00

(57) ABSTRACT

Cells comprising a heterologous nucleic acid sequence that encodes for a trehalase enzyme and optionally a heterologous nucleic acid sequence that encodes for a trehalose transporter protein are provided. Pharmaceutical compositions comprising the cells, methods of adoptive cell transfer comprising administering the cells and methods of determining suitability of a subject for the performance of the methods are also provided. A pharmaceutical composition comprising pharmaceutical grade trehalose is also provided, as is a kit comprising both compositions of the invention.

### CROSS REFERENCE TO RELATED APPLICATIONS

This application claims the benefit of priority of U.S. Provisional Patent Application No. 63/388,161 filed on Jul. 11, 2022, the contents of which are incorporated herein by reference in their entirety.

### REFERENCE TO AN ELECTRONIC SEQUENCE LISTING

The contents of the electronic sequence listing (HUJI-P-089-PCT.xml; Size: 20,340 bytes; and Date of Creation: Jul. 3, 2023) are herein incorporated by reference in their entirety.

### FIELD OF INVENTION

The present invention is in the field of adoptive cell transfer.

### BACKGROUND OF THE INVENTION

CD8 Cytotoxic T cells (CTLs) are the main adaptive-immune cells that play a key role in anti-cancer immunity. These cells have the ability to recognize and kill cells that express foreign or altered antigens, including tumor cells. Upon activation, CD8 T cells undergo a dramatic shift in cell metabolism switching from mitochondrial-based metabolism to aerobic glycolysis, to support their expansion and cytotoxic function. This transition known as the Warburg effect includes a shift in the primary energy production site to the cytosol while utilizing the mitochondria for rapid anabolism.

T cells rely on these metabolic changes to meet the demand for energy and biomass. Furthermore, T cell functionality and fate is governed by metabolic rewiring and therefore may be exploited for therapeutic purposes.

Adoptive T cell transfer therapy (ACT), a rapidly emerging immunotherapy approach, is based on the administration of ex vivo-activated and -expanded autologous tumor-specific CTLs. ACT harnesses the natural ability of T cells, to specifically recognize and eliminate target cells, and directs it to the treatment of advanced-stage cancers. ACT is currently one of the few immunotherapies that can induce objective clinical responses in significant numbers of patients with metastatic solid tumors.

ACT using autologous tumor-infiltrating lymphocytes (TILs) from resected metastatic tumor deposits has resulted in high response rates and reproducible robust responses in metastatic melanoma. However, application of TIL therapy has been limited to melanoma and only to cases where TILs can be retrieved from tumor deposits. To date, the foremost way to improve and extend the potency of TIL therapy is to administer peripheral T cells that have been genetically engineered to express tumor-specific antigen receptors. These receptors can be traditional ab-T cell receptors (TCRs), which recognize epitopes of intracellular antigens presented by MHC molecules, or chimeric antigen receptors (CARs). CARs are antibody single-chain variable fragments joined with TCR and T cell costimulatory receptor signaling domains, which recognize cell-surface antigens in a non-MHC-restricted manner. Both of these methods improve ACT potency by application of tumor restricted antigens or isolation of highly specific receptors against these targets.

Solid tumors build up a hostile microenvironment characterized by a continued reduction in O₂, glucose and other nutrients. Cancer cells subvert the metabolic characteristics of the tumor microenvironment to shape immune responses within tumors. Specifically, it has been shown that glycolysis within tumor cells cause depletion of extracellular glucose which restricts glucose availability to T cells. Decreased glucose availability causes suppression of glycolytic metabolism within T cells, and this is associated with dysfunction of infiltrating CTLs which immensely limits ACT and antiblockage immunotherapy of solid tumors. Methods and T cells that can overcome the glucose deprivation caused by the tumor are greatly needed.

### SUMMARY OF THE INVENTION

The present invention provides cells comprising a heterologous nucleic acid sequence that encodes for a trehalase enzyme and optionally nucleic acid sequence that encodes for a trehalose transporter protein. Pharmaceutical compositions comprising the cells, methods of adoptive cell transfer comprising administering the cells and methods of determining suitability of a subject for the performance of the methods are also provided. A pharmaceutical composition comprising pharmaceutical grade trehalose is also provided, as is a kit comprising both compositions of the invention.

According to a first aspect, there is provided a cell comprising a heterologous nucleic acid sequence that encodes for a trehalase enzyme.

According to some embodiments, the cell is a mammalian cell.

According to some embodiments, the mammal is a human.

According to some embodiments, the cell is an immune cell.

According to some embodiments, the immune cell is selected from a CD8 T cell, an NK cell, a tumor infiltrating lymphocyte (TIL), a macrophage, a dendritic cell, and a chimeric antigen receptor (CAR) expressing or recombinant T cell receptor expressing immune cell.

According to some embodiments, the trehalase enzyme is an insect trehalase.

According to some embodiments, the insect trehalase enzyme is drosophila melanogaster trehalase (TREH) or a functional fragment, variant, or homolog thereof.

According to some embodiments, the TREH comprises or consists of an amino acid sequence selected from SEQ ID NO: 1-3 or a fragment, variant, or homolog thereof comprising at least 85% sequence identity and comprising trehalase enzymatic function.

According to some embodiments, the TREH comprises or consists of SEQ ID NO: 1 or a fragment, variant or homolog thereof comprising at least 85% sequence identity and comprising trehalase enzymatic function.

According to some embodiments, the cell of the invention further comprises a heterologous nucleic acid sequence that encodes for a trehalose transporter protein.

According to some embodiments, the trehalose transporter protein is an insect protein.

According to some embodiments, the insect trehalose transporter protein is drosophila melanogaster trehalose transporter 1 (TRET1) or a functional fragment, variant or homolog thereof.

According to some embodiments, the TRET1 comprises or consists of an amino acid sequence selected from SEQ ID NO: 5-6 or a fragment, variant, or homolog thereof comprising at least 85% sequence identity and comprising trehalose transport activity.

According to some embodiments, the heterologous nucleic acid sequence encoding a trehalase enzyme is operatively linked to at least one regulatory element active in the cell.

According to some embodiments, the heterologous nucleic acid sequence encoding a trehalose transporter protein is operatively linked to at least one regulatory element active in the cell.

According to some embodiments, the regulatory element is constitutively active in the cell or is inducible to activity in the cell.

According to some embodiments, the cell has been extracted from a subject, grown in culture and made to express the heterologous nucleic acid sequence.

According to another aspect, there is provided a pharmaceutical composition comprising a cell of the invention and a pharmaceutically acceptable carrier, excipient or adjuvant.

According to some embodiments, the pharmaceutical composition of the invention is formulated for administration to a subject.

According to some embodiments, the pharmaceutical composition of the invention is formulated for systemic administration or administration to a tumor.

According to some embodiments, the pharmaceutical composition of the invention comprises trehalose.

According to some embodiments, the pharmaceutical composition of the invention comprises decreased glucose concentration as compared to cellular compositions comprising cells that cannot metabolize extracellular trehalose.

According to some embodiments, the pharmaceutical composition of the invention is for use in a method of adoptive cell therapy in a subject in need thereof.

According to another aspect, there is provided a method for adoptive cell therapy in a subject in need thereof, the method comprising administering to the subject a pharmaceutical composition of the invention and administering trehalose to the subject, thereby performing adoptive cell therapy.

According to some embodiments, the subject suffers from cancer.

According to some embodiments, the cancer is a solid cancer.

According to some embodiments, the solid cancer comprises a tumor microenvironment (TME) comprising reduced glucose concentration as compared to non-tumor regions within the subject.

According to some embodiments, the method further comprises before the administering confirming in the subject that a TME of the solid cancer comprises a glucose concentration below a predetermined threshold.

According to some embodiments, the predetermined threshold is the glucose concentration in the organ or bodily region in which the solid cancer is found in a healthy subject or a non-cancerous location in the organ or bodily region in the subject.

According to some embodiments, the cell is selected from: a CD8 T cell, an NK cell, a tumor infiltrating lymphocyte (TIL), a macrophage, a dendritic cell, and a chimeric antigen receptor (CAR) expressing or recombinant T cell receptor expressing immune cell.

According to some embodiments, the method further comprises receiving cells obtained from the subject, ex vivo genetically engineering the cells to express the heterologous nucleic acid sequences that encodes for a trehalase enzyme, and optionally the heterologous nucleic acid sequences that encodes for a trehalose transport protein and returning the genetically engineered cell to the subject.

According to some embodiments, the method further comprises activating the received cells before the genetic engineering.

According to some embodiments, the genetic engineering comprises infecting the cells with a virus comprising the heterologous nucleic acid sequence that integrates into the genome of the cell, or directly editing the genome of the cell with a genome editing protein or protein complex.

According to some embodiments, the trehalose is administered after, before or concomitantly with the pharmaceutical composition.

According to some embodiments, the administration of trehalose is continued until treatment of the subject is completed, is repeated every 2-5 days for 2 to 10 weeks, or both.

According to another aspect, there is provided a pharmaceutical composition comprising trehalose in pharmaceutical grade purity together with a pharmaceutically acceptable carrier.

According to some embodiments, the pharmaceutical composition comprises a high concentration of trehalose, wherein high is higher than the concentration used to treat neurodegenerative disease.

According to some embodiments, the pharmaceutical composition is for use in adoptive cell transfer therapy with a composition of the invention.

According to another aspect, there is provided a kit comprising a pharmaceutical composition of the invention comprising cells and a pharmaceutical composition of the invention comprising trehalose.

According to another aspect, there is provided a method of determining suitability of a subject suffering from a solid cancer to be treated by a method of the invention, the method comprising measuring glucose concentration in a TME of the solid cancer, wherein a glucose concentration below a predetermined threshold indicates the subject is suitable to treatment, thereby determining suitability of a subject.

According to some embodiments, the predetermined threshold is the glucose concentration in the organ or bodily region in which the solid cancer is found in a healthy subject or a non-cancerous location in the organ or bodily region in the subject.

According to some embodiments, the method further comprises performing a method of adoptive cell therapy of the invention on a suitable subject.

According to another aspect, there is provided a method of producing a cell of the invention, the method comprising:
a. providing a mammalian cell; and
b. introducing into the provided cell a nucleic acid sequence encoding for a trehalase enzyme to produce an engineered cell;
   thereby producing a cell of the invention.

According to some embodiments, the method further comprises introducing into the provided cell a nucleic acid sequence encoding for a trehalose transporter protein.

According to some embodiments, the method further comprises testing that the engineered cell is capable of surviving in the presence of trehalose and the absence or depletion of glucose.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1D: Expression of Drosophila Melanogaster Treh and Tret1 in transfected HEK-293T cells. HEK-293T cells were transfected with the indicated amount of HA-Tret1 and/or Tret1-3×Flag expression plasmids. Forty-eight hours post-transfection, cells were harvested, and cell extracts were subjected to immunoblot analysis. (1A) Immunoblot analysis of various amounts of HA-Treh using anti-HA antibody. (1B) Immunoblot analysis of various amounts of Tret1-3×Flag using anti-Flag. (1C) Same as in 1B with cells that were treated with 5 μg/ml brefeldin A for 5 hours before harvesting. (1D) Immunoblot analysis of HA-Tret1 and/or Tret1-3×Flag using anti-Flag and anti-HA antibodies. M—Molecular weight marker. E.V.—empty vector.

FIGS. 2A-2C: Treh and Tret1 co-expression rescues the proliferation and survival of HEK-293T cells in glucose-free, trehalose-supplemented medium. (2A) Histograms of cell survival as measured by PI staining using a flow cytometer. HEK-293T cells were transfected with either Tret1-3×Flag expression plasmid, HA-Treh expression plasmid or both together. 48 h post-transfection medium was replaced to a glucose-free medium supplemented with the indicated trehalose concentrations. Four days later flow analysis was performed. (2B) Histograms of cell survival as measured by PI staining using a flow cytometer. HEK-293T cells were co-transfected with Tret1-3×Flag and HA-Treh expression plasmids or pCDNA3.1 (empty vector). 48 h post-transfection medium was replaced with glucose-free medium supplemented with the 25 mM trehalose. Four days later, flow analysis was performed. (2C) Bar graph of MMT analysis. HEK-293T cells were transfected with pCDNA3.1 (empty vector), Tret1-3×Flag or HA-Treh expression plasmids or both plasmids together. 48 h post-transfection medium was replaced with glucose-free medium supplemented with 25 mM trehalose. Ten days later, cell survival was measured by MTT assay. Bar graph shows mean of 4 replicates (Unpaired t-test, mean±s.e.m, ***p<0.001).

FIG. 3: Treh and Tret1 co-expression allows utilization of Trehalose as a carbon source for glycolysis. Line graph of the Extra Cellular Acidification Rate (ECAR) of Tret1-3×Flag and Treh-GFP expressing (transduced with retrovirus expressing both Tret1-3×Flag and Treh-GFP) or wild-type HEK-293T cells. Trehalose was added at 5 mM, Oligomycin at 1 μM and 2DG (2-Deoxy-D-glucose) at 50 mM. Each point shows mean of 12 replicates (Unpaired t-test, mean±s.e.m, ***p<0.001).

FIG. 4: Treh and Tret1 co-expression rescue the proliferation and survival of human T cell line (Jurkat) in glucose-free, trehalose supplemented medium. Bar graphs of MTT analysis. Jurkat cells were transduced with retrovirus expressing both Tret1-3×Flag and Treh-GFP, or GFP alone. Cells were then sorted by FACS for GFP positive cells and were grown for a week in a glucose-free medium (Red circle), glucose-free medium supplemented with 10 mM Glucose (Blue square) or glucose-free medium supplemented with 5 mM trehalose (Green triangle). Cell-survival and proliferation were then analyzed by MTT assay. Bar graph shows mean of 6 replicates (Unpaired t-test, mean±s.d, **p<0.01).

FIGS. 5A-5D: DmTret1-1B can substitute Tret1. (5A) FACS overlay histogram of Tret1-1B-Flag×3 expression in Jurkat cells transduced with empty or DmTret1-1B-Flag×3-expressing lentivirus. (5B) ECAR measured by Seahorse analysis following trehalose or glucose and 2DG injections of empty vector or two independently transduced clones (replicates) of Tret1-1B/Treh-expressing Jurkat cells. (5C) Bar graph of MTT assay. Empty virus or Tret1-1B/Treh-expressing Jurkat cells were grown for three days in 3 different media, glucose-containing medium, glucose-free and trehalose-supplemented medium, or glucose-free medium. Then, viability of the cells was assessed by MTT assay. (5D) A bar graph summarizing the percentages of GFP/RFP expressing cells at various time points. NK92 cells were transduced with GFP and RFP (empty virus) or DmTret1-1B GFP and Treh-HA RFP expressing lentiviruses and were grown in glucose-free and trehalose-supplemented media or normal glucose containing media for 28 days. Cells were analyzed for expression of GFP and RFP at the indicated time points by FACS.

FIGS. 6A-6I: Human primary T cells utilize trehalose for glycolysis, proliferation and cytokine production. Human primary PBMCs were activated with anti-CD3 and anti-CD28 for 48 hours. Then, they were retrovirally transduced with Treh1 and a trehalose transporter. (6A) FACS analysis of the T cells transduced with Treh1-GFP and Dm-Tret1-1B-mKate2. CD8 T cells were gated, and expression was analyzed 48 h after transduction. (6B) FACS analysis of the T cells transduced with Treh1-GFP and Dm-Tret1-1A-mKate2. CD8 T cells were gated, and expression was analyzed 48 h after transduction. (6C) Normalized ECAR measured by Seahorse analysis following trehalose or glucose supplementation and oligomycin and 2DG injections of Treh1 or DmTret1-1B/Treh1-expressing CD8 T cells. (6D-G): DmTret1-1B/Treh-expressing human CD8 T cells were incubated for 24 h with 13C6-Glucose or 13C12-Trehalose. Then cell extracts and media were subjected to metabolic analysis using LC-MS. (6D) Levels of lactate isotopologues in the medium of the cells. (6E-G) Isotopologue distributions in the cell fraction of detected key metabolites generated during (6E) glycolysis, (6F) the TCA cycle and (6G) the phosphate pentose pathway. (6H) Treh1 or DmTret1-1B/Treh1-expressing CD8 T cells were grown in 3 different media: glucose and trehalose, trehalose, or glucose free medium for 10 days. Cells were counted every 3-4 days. (6I) Untransduced or DmTret1-1B/Treh1-expressing T cells were restimulated with anti-CD3 for 18 hours in 3 different media. Media was collected and INF-γ concentration was measured by ELISA at optical density of 605 (O.D.605).

### DETAILED DESCRIPTION OF THE INVENTION

The present invention, in some embodiments, provides cells comprising a heterologous nucleic acid sequence that encodes for a trehalase enzyme. Pharmaceutical compositions comprising the cells, methods of adoptive cell transfer comprising administering the cells and methods of determining suitability for the performance of the methods are also provided. A pharmaceutical composition comprising pharmaceutical grade trehalose is also provided, as is a kit comprising both compositions of the invention.

The inventors have engineered CTLs that are able to use trehalose as a carbon source instead of glucose. Trehalose is a highly stable, non-toxic disaccharide formed by a 1,1-glycosidic bond between two α-glucose units. Human T cells, as well as most other human cells, do not have the genes that allow them to uptake and catabolize intracellular trehalose. The trehalase gene (Treh) is present in humans but is a secreted protein not present in the cytoplasm for the purposes of sugar catabolism. Further, it is not expressed in most cells (including immune cells). SLC2A8 (also called GLUT8) is a known human sugar transporter that has been shown to transport trehalose into human cells, however, this protein is also not expressed by most cell types and its relative efficiency of transport is not well characterized. Therefore, the inventors introduced both the trehalose transporter (Tret1) and the trehalose-hydrolyzing enzyme (Trehalase-Treh) from insects into human cells. Treh is a highly efficient treahlase that catabolizes the trehalose into two glucose molecules. Its expression alone was sufficient to induce survival of mammalian cells in a glucose-free, trehalase-supplemented medium, likely due to sufficient uptake of trehalose by a mammalian transporter. Tret1 is a high-affinity trehalose membrane-localized transporter that allowed for the efficient uptake of trehalose into the cytoplasm of human cells. It was found to be significantly superior to the human transporter alone and greatly improved survival when trehalose was the only sugar source. The glucose produced from trehalose can be used as a substrate in the glycolysis pathway, and therefore these engineered cells can maintain their glucose-depending metabolism that is critical to survival and function (e.g., their effector and anti-tumor functions in immune cells).

### Cells

By a first aspect, there is provided a cell comprising a heterologous nucleic acid sequence that encodes for an enzyme capable of converting a non-canonical carbon source into a canonical carbon source.

By another aspect, there is provided a cell comprising a heterologous nucleic acid sequence that encodes for a trehalase enzyme.

As used herein, the term “non-canonical carbon source” refers to an organic source being a carbohydrate, amino acid, fatty acid or glycerol that is used by anchemoheterotrophs organism as a source of energy. A “canonical carbon source” is therefore an organic source that a human use for a source of energy. The non-canonical carbon sources of the inventions are those used by organism other than human cells such as those used by insects, and chemoheterotrophs bacteria, protozoa and fungi. A canonical carbon source is thus for example, glucose, fructose, dextrose, sucrose and the like. In some embodiments, the canonical carbon source is glucose. Non-limiting examples of such non-canonical carbon sources are trehalose (utilized by insects), cellobiose, gentiobiose, sophorose, isomaltose, and laminaribiose and the like. In some embodiments, the non-canonical carbon source is trehalose.

In some embodiments, converting a non-canonical carbon source into a canonical carbon source is catabolizing the non-canonical carbon source. In some embodiments, an enzyme capable of converting trehalose is trehalase. In some embodiments, an enzyme capable of converting cellobiose, gentiobiose, and/or sophorose is beta-glucosidase. In some embodiments, a beta-glucosidase mRNA is found in the accession number AF317840. In some embodiments, an enzyme capable of converting isomaltose is isomaltase. In some embodiments, an isomaltase mRNA is found in the accession number NM_001041. In some embodiments, an enzyme capable of converting laminaribiose is laminarinase. In some embodiments, a laminaribiose mRNA is found in the accession number KY29026.1.

In some embodiments, the cell is a mammalian cell. In some embodiments, the cell is not an insect cell. In some embodiments, the mammal is a human. In some embodiments, the cell is a cell in culture. In some embodiments, the cell is an ex vivo cell. In some embodiments, the cell is an in vitro cell. In some embodiments, the cell is from a subject. In some embodiments, the cell was extracted from a subject. In some embodiments, the cell is a population of cells. In some embodiments, the population is an in vitro expanded population. In some embodiments, the cell was grown in culture. In some embodiments, the cell is in culture. In some embodiments, the cell was made to express the heterologous nucleic acid sequence.

In some embodiments, the cell does not comprise an endogenous trehalase gene. In some embodiments, the cell does not comprise endogenous trehalase expression. In some embodiments, expression is cytoplasmic expression. In some embodiments, expression is intracellular expression. In some embodiments, expression is not secretion. In some embodiments, the cell expresses an endogenous trehalose transporter protein. In some embodiments, the endogenous trehalose transporter protein is SLC2A8 (also called GLUT8). In some embodiments, expressing is expressing at a level sufficient to transport extracellular trehalose into the cytoplasm of the cell.

In some embodiments, the cell is a therapeutic cell. In some embodiments, the cell is a cell that is used in adoptive cell transfer. In some embodiments, the cell is a cell that is transferred to a subject to produce a therapeutic result. In some embodiments, the cell is an immune cell. In some embodiments, the immune cell is a cytotoxic immune cell. In some embodiments, an immune cell is selected from a T cell, a B cell, a natural killer (NK) cell, a tumor infiltrating lymphocyte (TIL), a macrophage, a dendritic cell and a modified immune cell. In some embodiments, an immune cell is selected from a T cell, a B cell, a natural killer (NK) cell, a macrophage, and a dendritic cell. In some embodiments, the immune cell is a TIL. In some embodiments, the immune cell is selected from a T cell, a natural killer (NK) cell, a macrophage, and a dendritic cell. In some embodiments, the immune cell is a T cell. In some embodiments, the T cell is selected from a CD8 T cell and a CD4 T cell. In some embodiments, the T cell is a CD8 T cell. In some embodiments, a CD8 T cell is a cytotoxic T cell. In some embodiments, the T cell is a CD4 T cell. In some embodiments, a CD4 T cell is a T helper cell. In some embodiments, the immune cell is selected from a CD8 T cell and an NK cell. In some embodiments, the immune cell is an NK cell. In some embodiments, the immune cell is a modified immune cell. In some embodiments, the modified immune cell is a chimeric antigen receptor (CAR) expressing immune cell. In some embodiments, the CAR cell is a CAR-T cell. In some embodiments, the CAR cell is a CAR-NK cell. In some embodiments, the modified immune cell is a recombinant T cell receptor (rTCR) expressing immune cell. In some embodiments, the rTCR is a rTCR-T cell. In some embodiments, the rTCR is a rTCR-NK cell.

As used herein, the terms “CAR-T cell” and “CAR-NK cell” refer to an engineered receptor which has specificity for at least one protein of interest (for example an immunogenic protein with increased expression following treatment with an epigenetic modifying agent) and is grafted onto an immune effector cell (a T cell or NK cell). In some embodiments, the CAR-T cell has the specificity of a monoclonal antibody grafted onto a T-cell. In some embodiments, the CAR-NK cell has the specificity of a monoclonal antibody grafted onto a NK-cell. In some embodiments, the T cell is selected from a cytotoxic T lymphocyte and a regulatory T cell.

CAR-T and CAR-NK cells and their vectors are well known in the art. Such cells target and are cytotoxic to the protein for which the receptor binds. In some embodiments, a CAR-T or CAR-NK cell targets at least one viral protein. In some embodiments, a CAR-T or CAR-NK cell targets a plurality of viral proteins. In some embodiments, a CAR-T or CAR-NK cell targets a viral protein with increased expression due to contact with an epigenetic modifying agent.

Construction of CAR-T cells is well known in the art. In one non-limiting example, a monoclonal antibody to a viral protein can be made and then a vector coding for the antibody will be constructed. The vector will also comprise a costimulatory signal region. In some embodiments, the costimulatory signal region comprises the intracellular domain of a known T cell or NK cell stimulatory molecule. In some embodiments, the intracellular domain is selected from at least one of the following: CD3Z, CD27, CD28, 4-1BB, OX40, CD30, CD40, PD-1, ICOS, lymphocyte function-associated antigen-1 (LFA-1), CD2, CD 7, LIGHT, NKG2C, B7-H3, and a ligand that specifically binds with CD83. In some embodiments, the vector also comprises a CD3Z signaling domain. This vector is then transfected, for example by lentiviral infection, into a T-cell.

rTCRs are well known in the art and any recombinant T cell receptor may be used and expressed in the cell. Methods of designing and constructing rTCRs are also well known and can be found for example in Jones et al., “Empirical and Rational Design of T Cell Receptor-Based Immunotherapies”, Front. Immunol., 2021 January; 11:585385, herein incorporated by reference in its entirety.

In some embodiments, the immune cells are activated immune cells. In some embodiments, the T cells are activated T cells. In some embodiments, activated is ex vivo activated. The “ex vivo activation”—in the context of the present invention refers to ex vivo activation manifested in increase activity of non-engineered cells without increase of their number, in genetic manipulation changing proteins expressed by the cells, in expansion of cell number or a combination of the above. Activation can be used for the purpose of increase in cell number, improved affinity to tumor cells, and increase in tumor damaging activities or combinations of two or more of the above. The activation may be merely by ex vivo exposure to agents such as antibodies, ionophores, cytokines, hormones, metabolites, pathogen-associated molecular pattern molecules (PAMPs), peptides, neurotransmitters or neuropeptides. In some embodiments, the activation is by anti-CD3 antibody.

The activation may also be by genetic manipulations-causing the immune cells to express proteins that improve their cancer recognitions and/or cancer destroying properties. Examples of such manipulations are for creation of tumor infiltrating lymphocytes (TILs), chimeric antigen receptor (CAR-T cells) and recombinant T cell receptor (TCR-T cells). For example, peripheral T cells are genetically engineered to express tumor-specific antigen receptors. That can be traditional ab-T cell receptors (TCRs), which recognize epitopes of intracellular antigens presented by MHC molecules, or chimeric antigen receptors (CARs). CARs are antibody single-chain variable fragments joined with TCR and T cell costimulatory receptor signaling domains, which recognize cell-surface antigens in a non-MHC-restricted manner.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

In some embodiments, the heterologous nucleic acid sequence is comprised in a heterologous nucleic acid molecule. In some embodiments, the heterologous nucleic acid sequence is integrated into the cell's genome. In some embodiments, a heterologous nucleic acid molecule is integrated into the cell's genome. The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, circulating nucleic acids, fragments of genomic DNA or RNA, degraded nucleic acids, ribozymes, viral RNA or DNA, nucleic acids of infectios origin, amplification products, modified nucleic acids, plasmidical or organellar nucleic acids and artificial nucleic acids such as oligonucleotides. In some embodiments, the nucleic acid molecule is a DNA molecule. In some embodiments, the nucleic acid molecule is an expression vector. In some embodiments, the nucleic acid molecule comprises a cDNA. In some embodiments, the cDNA is devoid of introns. In some embodiments, the cDNA encodes for the trehalase. In some embodiments, the cDNA encodes for the trehalose transporter. In some embodiments, the nucleic acid molecule comprises an open reading frame. In some embodiments, the nucleic acid molecule comprises a coding region. In some embodiments, the open reading frame or coding region encodes the trehalase. In some embodiments, the open reading frame or coding region encodes the trehalose transporter.

In some embodiments, the trehalase is soluble trehalase. In some embodiments, the trehalase is cytoplasmic trehalase. In some embodiments, the trehalase is not membrane trehalase. In some embodiments, the trehalase is not secreted trehalase. In some embodiments, the trehalase does not comprise a transmembrane domain. In some embodiments, the trehalase does not comprise a signal peptide. In some embodiments, the trehalase is a non-mammalian trehalase. In some embodiments, the trehalase is an insect trehalase. In some embodiments, the insect is a fly. In some embodiments, the fly is drosophila. In some embodiments, the insect is a bee. In some embodiments, the trehalase is an insect homolog of fly trehalase. In some embodiments, the drosophila is drosophila melanogaster. In some embodiments, the D. melanogaster trehalase gene is Treh. In some embodiments, D. melanogaster Treh is provided in entrez gene 45368. In some embodiments, the heterologous nucleic acid molecule comprises a Treh cDNA. In some embodiments, the cDNA is the DNA version of a Treh mRNA. In some embodiments, the D. melanogaster trehalase is selected from transcript variants A, B, C, D, E, F and G. In some embodiments, the trehalase is encoded by transcript variant A (accession number NM_166421). In some embodiments, the trehalase is encoded by transcript variant B (accession number NM_166425). In some embodiments, the trehalase is encoded by transcript variant C (accession number NM_166423). In some embodiments, the trehalase is encoded by transcript variant D (accession number NM_080082). In some embodiments, the trehalase is encoded by transcript variant E (accession number NM_166422). In some embodiments, the trehalase is encoded by transcript variant F (accession number NM_166424). In some embodiments, the trehalase is encoded by transcript variant G (accession number NM_001274186). In some embodiments, the coding region from one of transcript variants A, B, C, D, E, F and G is used in the nucleic acid molecule. It will be understood by a skilled artisan that the untranslated regions of the mRNA need not be included in the nucleic acid molecule. In some embodiments, the trehalase coding region comprises or consists of the nucleotide sequence

(SEQ ID NO: 4)
ATGGCCTCTCCAGCGAATCCATCGAGCAATCACAAAATGAACGGAAATGG
TAAAATCTACTGCGAGGGCAATCTGCTGCACACCATCCAAACGGCAGTGC
CCAAACTATTTGCGGATTCGAAAACGTTTGTGGACATGAAGCTGAACAAT
TCGCCCGACAAGACCCTCGAGGACTTTAATGCCATGATGGAGGCCAAGAA
TCAGACGCCAAGCAGTGAGGATCTCAAGCAGTTTGTCGATAAGTACTTCA
GTGCACCGGGCACCGAGCTTGAGAAATGGACGCCCACCGACTGGAAGGAG
AATCCCAGTTTCCTCGACCTGATCTCCGACCCAGATCTGAAGCAATGGGG
CGTCGAGCTGAATAGCATTTGGAAGGACTTGGGACGCAAAATGAAGGACG
AGGTGTCAAAGAATCCCGAATACTACTCAATCATTCCCGTGCCAAATCCA
GTGATCGTGCCCGGCGGTCGTTTCATTGAGTTCTACTACTGGGACTCCTA
CTGGATCATCCGTGGACTTCTCTACAGCCAGATGTTTGACACCGCGCGCG
GCATGATTGAGAACTTCTTTTCCATTGTCAATCGGTTCGGTTTTATTCCA
AACGGCGGTCGAGTCTACTACCACGGTCGCTCCCAGCCGCCACTTCTAAC
CGGTATGGTCAAGTCGTACGTGGACTTCACCAACGATGACAAGTTCGCCA
TCGATGCCCTGGACACGCTGGAGCACGAGTTCGAGTTCTTTGTGAACAAC
CACAATGTCACGGTGAAGAATCACAGCCTGTGTGTGTACCGCGATTCGTC
GTCCGGACCGCGACCAGAATCCTACCGAGAGGATGTGGAGACCGGCGAGG
AGTTCCCCACGGATGAGGCCAAGGAACTGCATTACAGTGAACTCAAGGCA
GGCGCCGAATCGGGCATGGACTTTAGCTCGCGCTGGTTCATCTCACCGAC
TGGAACCAATGATGGCAACCGGAGCGCTCTGAGCACCACCTCCATTGTGC
CCGTCGACCTGAATGCCTATCTCTACTGGAACGCCAAGTTGATTGCCGAG
TTCCATTCCAAAGCGGGCAACACCAAAAAGGTCACCGAATACGAGACCAA
GGCCGAGAAACTCCTTCTGGGTATCCAAGAAGTTTTGTGGAACGAGGAGG
CCGGTGTCTGGTTGGACTACGATATGATTAACCAGAAGCCTCGCGATTAC
TACACGCCCACCAATCTATCTCCACTGTGGGTGAAGGCCTTCAACATTTC
GGAGTCCGAAAAGATATCGGCTTCGGTTATGGCCTACATTGAGAGGAACA
AGCTGGACAGCTTCCCTGGCGGAGTTCCCAACACGCTGAGCTACACCGGA
GAACAGTGGGATGCCCCCAATGTGTGGGCACCGATGCAGTACATCCTGGT
CGAGGGCCTAAACAACCTGAACACTCCCGAGGCCAAGAATATGTCACTGA
AGTGGGCCACCAGGTGGGTGAAGACAAACTTTGCGGCGTTTAGCAAGGAC
AGGCACATGTACGAGAAGTACAACGCCGATGAGTTCGGAGTTGGAGGCGG
CGGTGGCGAGTACGAGGTACAGACTGGATTCGGTTGGTCCAACGGTGTGA
TCATCGAGTGGCTGAGCAAGCACGGGCGCGACATTTCCATTGGATCCGGT
TGTGGCTGCCTAGCAGGTGAAAAGCGGCAGTAG.

In some embodiments, D. melanogaster trehalose enzyme (TREH) is selected from protein isoforms A, B, C, D, E, F and G. In some embodiments, the trehalase is trehalose isoform A, D, E or G (accession number NP_726023, NP_524821, NP_726024, and NP_001261115 SEQ ID NO: 1). In some embodiments, the trehalase is trehalose isoform G. In some embodiments, the trehalase is trehalose isoform B (accession number NP_726027, SEQ ID NO: 2). In some embodiments, the trehalase is trehalose isoform C or F (accession number NP_726025, or NP_726026 SEQ ID NO: 3). In some embodiments, the trehalase comprises or consists of the amino acid sequence MASPANPSSNHKMNGNGKIYCEGNLLHTIQTAVPKLFADSKTFVDMKLNNSPDKT LEDFNAMMEAKNQTPSSEDLKQFVDKYFSAPGTELEKWTPTDWKENPSFLDLISDP DLKQWGVELNSIWKDLGRKMKDEVSKNPEYYSIIPVPNPVIVPGGRFIEFYYWDSY WIIRGLLYSQMFDTARGMIENFFSIVNRFGFIPNGGRVYYHGRSQPPLLTGMVKSYV DFTNDDKFAIDALDTLEHEFEFFVNNHNVTVKNHSLCVYRDSSSGPRPESYREDVE TGEEFPTDEAKELHYSELKAGAESGMDFSSRWFISPTGTNDGNRSALSTTSIVPVDL NAYLYWNAKLIAEFHSKAGNTKKVTEYETKAEKLLLGIQEVLWNEEAGVWLDYD MINQKPRDYYTPTNLSPLWVKAFNISESEKISASVMAYIERNKLDSFPGGVPNTLSY TGEQWDAPNVWAPMQYILVEGLNNLNTPEAKNMSLKWATRWVKTNFAAFSKDR HMYEKYNADEFGVGGGGGEYEVQTGFGWSNGVIIEWLSKHGRDISIGSGCGCLAG EKRQ (SEQ ID NO: 1). In some embodiments, the TREH comprises or consists of an amino acid sequence selected from SEQ ID NO: 1-3.

In some embodiments, the trehalase is a trehalase homolog. In some embodiments, a trehalase homolog comprises at least 70, 75, 80, 85, 90, 95, 97, 99 or 100% sequence identity to SEQ ID NO: 1. Each possibility represents a separate embodiment of the invention. In some embodiments, a trehalase homolog comprises at least 85% sequence identity to SEQ ID NO: 1. In some embodiments, a trehalase homolog comprises trehalase activity. In some embodiments, a trehalase homolog comprises glycoside hydrolase activity. In some embodiments, a trehalase homolog comprises the ability to metabolize trehalose. In some embodiments, a trehalase homolog comprises the ability to convert trehalose into glucose. In some embodiments, a trehalase homolog comprises the ability to convert trehalose into two molecules of glucose. The functional domain within TREH has been well characterized and is known in the art (see for example Tellis, et al., “Evolutionary and structure-function analysis elucidates diversification of prokaryotic and eukaryotic trehalases”, J. Biomol. Struct. Dyn., 2019, July; 37(11):2926-2937, and Yoshida et al., “Molecular characterization of Tps1 and Treh genes in Drosophila and their role in body water homeostasis”, Sci. Rep., 2016 Jul. 29; 6:30582, herein incorporated by reference in their entireties.). Within D. melanogaster trehalase the trehalase domain (amino acids 71 to 578 of SEQ ID NO: 1) has been found to be critical to trehalase activity. Conservative mutations in this domain (e.g., a basic amino acid substituted for basic and the like) will still result in a protein having trehalase activity. Most non-conserved mutation in this domain will not result in a protein with trehalase activity. Outside of this domain most mutations will not affect trehalase activity.

In some embodiments, the cell further comprises a heterologous nucleic acid sequence that encodes for a non-canonical carbon source transport protein. In some embodiments, the cell further comprises a heterologous nucleic acid sequence that encodes for a trehalose transporter protein. In some embodiments, the cell comprises a nucleic acid molecule comprising the heterologous nucleic acid sequence encoding a trehalose transporter protein. In some embodiments, a trehalose transporter protein is a trehalose transporter. In some embodiments, a single nucleic acid molecule comprises both the sequence encoding a trehalase and the sequence encoding the trehalose transporter. In some embodiments, the single nucleic acid molecule is the cells genome. In some embodiments, the single nucleic acid molecule is bicistronic molecule. In some embodiments, the bicistronic molecule is a bicistronic retrovirus.

In some embodiments, the trehalose transporter is a non-mammalian trehalose transporter. In some embodiments, the trehalose transporter is an insect trehalose transporter. In some embodiments, the insect is a fly. In some embodiments, the insect is a bee. In some embodiments, the fly is drosophila. In some embodiments, the drosophila is drosophila melanogaster. In some embodiments, the trehalose transporter is a homolog of fly transporter. In some embodiments, the D. melanogaster trehalose transporter gene is Tret1. In some embodiments, D. melanogaster Tret1 is provided in entrez gene 36248. In some embodiments, Tret1 is Tret1-1. In some embodiments, the heterologous nucleic acid molecule comprises a Tret1 cDNA. In some embodiments, the cDNA is the DNA version of a Tret1 mRNA. In some embodiments, the D. melanogaster trehalose transporter is selected from transcript variants A and B. In some embodiments, the trehalose transporter is encoded by transcript variant A (accession number NM_136849). In some embodiments, the trehalose transporter is encoded by transcript variant B (accession number NM_165845). In some embodiments, the coding region from transcript variants A or B is used in the nucleic acid molecule. It will be understood by a skilled artisan that the untranslated regions of the mRNA need not be included in the nucleic acid molecule. In some embodiments, the Tret1 variant A coding region comprises or consists of the nucleotide sequence atgagtggacgcgacaacagaggagccggcggcggaggcggcggtcatcaaccgctgagcaacgccatgggcaagctcaag gaaaaactaacccgagtaggtgacgagctgggctatcatcgtgtggagagcaatttgtccacatcgaacacagcgaccagtttgga cacaattctgccagaggatccgttcctattcccccaagtgtcaccccaaaggcatccacaaaatacagtaaggacacagcgtttgctg gaggacgagccaccgctatcgtttcgcccgcttttggaggacgacgacatcaacgagccacctacacagcagcaacagaggactc ccctgcgcgccagcggtagtttggagttgactcccttgccaccgccgcccacatcattggagatacgtgagcatcgcgatcgccag cagagaggtgcccagggtgacgagctgcagcgcagcaagcagagcttgaaaggatcacgcgtgtccttcgagcgacgggatac cggaaacagcaataccaacagtaacaaagcagcggagagcagcgacgaggacagtttcgaggagaagcgaactggtttccagc aacagaaggcaaccagcgtcgatcacaagggcatcttgaaggacctgaagcacattttggccaacgacaatcgccggcagtttca ggccaagaagcacgtatccctggacgttaagggcacacgcttcctgcaggatctactgaaggaatcgtcateggaagaggagttcc acaagacacggcgggagttccagggtcgcaagcaccagagtttggatccacgtgtcacattcaagctcgacaaagtcctccaggg atcgagtaccgacagtgacgaggagggcgaagatgccgagcacaaacgcctgatccatcgacccaaggacattaccaagccggt gatcatcgatcttaaggatctggaaagcgagagcgacgaggactttctcacatcccgtcagcacttccaacagcagcgttccatcag cacagattcccgcaagagccggcgactctacgagatggacgagatggacaacaaacgcggcgagaacatccgtcatgcggtgc ccttcgtgcgccagataaccgaggatggcaagcccaagctggaggtctaccgtcccacaacgaatcccatatacatttggacacag gtcttggctgctctgagtgtctcgctgggctccctagtggtcggatttgtcagcgcttacacgtcgcctgctcttgtgtcgatgaccgat cggaatatcacctcgtttgaggtcacccaagatgctggttcttgggttggtggcatcatgccgctggctggattggcaggtggcatag ccggaggacccttgatcgaatatctgggcaggcgcaacaccatcctggccactgcggtgcccttcattgtctcatcactactgattgc ctgtgctgtgaatgtggctatggttctgtgcggacgattcctggctggattctgcgttggcattgcctctttatccctgcccgtctacctg ggtgaaactgtgcagcctgaggtgcgaggcactttgggtttgctgcccacggcctttggtaacataggcatcctgctctgcttcgtgg cgggatcctttatgaattggtcgatgctggccttcttgggagctgctctgcccgttcccttcctgattttaatgttccttattceggaaactc cgcgctggtttgtgggccgtggtctggaggagcgcgccaggaaggctttgaaatggctgcgtggcaaggaggctgatgtggagc ccgaactgaagggtttgatgcgatcccaggccgatgccgatcgtcaggcatcgcgaaacaccatgttggagctgcttaagctcaac aatctgaagccactatcaatttccttgggtctgatgttcttccaacagttcagcggtatcaacgcggttatcttctacacggtccagatctt caaggatgcgggatccaccattgatggcaatctctgcacgatcattgtgggaattgttaactttttggctacgtttataggcatcgttctg attgatcggggggcagaaagattcttctatatgtctccgacatcgccatggttctgaccctgttcgttctgggcggcttcttctactgca agacgtacggtccggatgtgtcccatttgggttggttgccactcacttgcttcgtcatctacattctgggattctccctgggctteggacc cattccctggctgatgatgggtgagattctgccggccaaaattcgcggatcggctgcctcggtggccacggctttcaactggttctgc acgtttgtggtgaccaagaccttccaggacctcacggttgctatgggcgcgcatggagegttctggctatteggegccatctgcttcg tgggtctgttctttgtgataatctacgtgcccgaaacgcagggcaagacgctggaggacatcgagcggaagatgatgggccgtgtg cgccgcatgtcctcggtggccaatatcaagccattgtccttcaacatgtaa (SEQ ID NO: 7). In some embodiments, the Tret1 variant B coding region comprises or consists of the nucleotide sequence atgaagatcctgatgcgagcggacacccatgtgtccttctccgtgcccgtcgaggagcccaaggccatttgtaccttctcccaggtct tggctgctctgagtgtctcgctgggctccctagtggtcggatttgtcagcgcttacacgtcgcctgctcttgtgtcgatgaccgatcgg aatatcacctcgtttgaggtcacccaagatgctggttcttgggttggtggcatcatgccgctggctggattggcaggtggcatagccg gaggacccttgatcgaatatctgggcaggcgcaacaccatcctggccactgeggtgcccttcattgtctcatcactactgattgcctgt gctgtgaatgtggctatggttctgtgcggacgattcctggctggattctgcgttggcattgcctctttatccctgcccgtctacctgggtg aaactgtgcagcctgaggtgcgaggcactttgggtttgctgcccacggcctttggtaacataggcatcctgctctgcttcgtggcggg atcctttatgaattggtcgatgctggccttcttgggagctgctctgcccgttcccttcctgattttaatgttccttattceggaaactcegcg ctggtttgtgggccgtggtctggaggagcgcgccaggaaggctttgaaatggctgcgtggcaaggaggctgatgtggagcccgaa ctgaagggtttgatgcgatcccaggccgatgccgatcgtcaggcatcgcgaaacaccatgttggagctgcttaagctcaacaatctg aagccactatcaatttccttgggtctgatgttcttccaacagttcageggtatcaacgcggttatcttctacacggtccagatcttcaagg atgcgggatccaccattgatggcaatctctgcacgatcattgtgggaattgttaactttttggctacgtttataggcatcgttctgattgat cgggcgggcagaaagattcttctatatgtctccgacatcgccatggttctgaccctgttcgttctgggcggcttcttctactgcaagacg tacggtccggatgtgtcccatttgggttggttgccactcacttgcttcgtcatctacattctgggattctccctgggctteggacccattcc ctggctgatgatgggtgagattctgccggccaaaattcgcggatcggctgcctcggtggccacggctttcaactggttctgcacgttt gtggtgaccaagaccttccaggacctcacggttgctatgggcgcgcatggagcgttctggctattcggcgccatctgcttcgtgggt ctgttctttgtgataatctacgtgcccgaaacgcagggcaagacgctggaggacatcgagcggaagatgatgggccgtgtgcgcc gcatgtcctcggtggccaatatcaagccattgtccttcaacatgtaa (SEQ ID NO: 8). In some embodiments, SEQ ID NO: 7 encodes SEQ ID NO: 5. In some embodiments, SEQ ID NO: 8 encodes SEQ ID NO: 6.

In some embodiments, D. melanogaster trehalose enzyme (TRET1) is selected from protein isoforms A and B. In some embodiments, the trehalose transporter is trehalose isoform A (accession number NP_610693, SEQ ID NO: 5). In some embodiments, TRET1-1A (isoform A) is the long isoform comprising 857 amino acids and a molecular weight of about 97 kDa. In some embodiments, the trehalose transporter is trehalose isoform B (accession number NP_725068, SEQ ID NO: 6). In some embodiments, TRET1-1B (isoform B) is the short isoform comprising only 489 amino acids. In some embodiments, the TRET1 comprises or consists of an amino acid sequence selected from SEQ ID NO: 5-6. In some embodiments, the TRET1 comprises or consists of an amino acid sequence MSGRDNRGAGGGGGGHQPLSNAMGKLKEKLTRVGDELGYHRVESNLSTSNTATS LDTILPEDPFLFPQVSPQRHPQNTVRTQRLLEDEPPLSFRPLLEDDDINEPPTQQQQR TPLRASGSLELTPLPPPPTSLEIREHRDRQQRGAQGDELQRSKQSLKGSRVSFERRDT GNSNTNSNKAAESSDEDSFEEKRTGFQQQKATSVDHKGILKDLKHILANDNRRQFQ AKKHVSLDVKGTRFLQDLLKESSSEEEFHKTRREFQGRKHQSLDPRVTFKLDKVLQ GSSTDSDEEGEDAEHKRLIHRPKDITKPVIIDLKDLESESDEDFLTSRQHFQQQRSIST DSRKSRRLYEMDEMDNKRGENIRHAVPFVRQITEDGKPKLEVYRPTTNPIYIWTQV LAALSVSLGSLVVGFVSAYTSPALVSMTDRNITSFEVTQDAGSWVGGIMPLAGLAG GIAGGPLIEYLGRRNTILATAVPFIVSSLLIACAVNVAMVLCGRFLAGFCVGIASLSL PVYLGETVQPEVRGTLGLLPTAFGNIGILLCFVAGSFMNWSMLAFLGAALPVPFLIL MFLIPETPRWFVGRGLEERARKALKWLRGKEADVEPELKGLMRSQADADRQASR NTMLELLKLNNLKPLSISLGLMFFQQFSGINAVIFYTVQIFKDAGSTIDGNLCTIIVGI VNFLATFIGIVLIDRAGRKILLYVSDIAMVLTLFVLGGFFYCKTYGPDVSHLGWLPL TCFVIYILGFSLGFGPIPWLMMGEILPAKIRGSAASVATAFNWFCTFVVTKTFQDLT VAMGAHGAFWLFGAICFVGLFFVIIYVPETQGKTLEDIERKMMGRVRRMSSVANIK PLSFNM (SEQ ID NO: 5). In some embodiments, the TRET1 comprises or consists of an amino acid sequence

(SEQ ID NO: 6)
MKILMRADTHVSFSVPVEEPKAICTFSQVLAALSVSLGSLVVGFVSAYTS
PALVSMTDRNITSFEVTQDAGSWVGGIMPLAGLAGGIAGGPLIEYLGRRN
TILATAVPFIVSSLLIACAVNVAMVLCGRFLAGFCVGIASLSLPVYLGET
VQPEVRGTLGLLPTAFGNIGILLCFVAGSFMNWSMLAFLGAALPVPFLIL
MFLIPETPRWFVGRGLEERARKALKWLRGKEADVEPELKGLMRSQADADR
QASRNTMLELLKLNNLKPLSISLGLMFFQQFSGINAVIFYTVQIFKDAGS
TIDGNLCTIIVGIVNFLATFIGIVLIDRAGRKILLYVSDIAMVLTLFVLG
GFFYCKTYGPDVSHLGWLPLTCFVIYILGFSLGFGPIPWLMMGEILPAKI
RGSAASVATAFNWFCTFVVTKTFQDLTVAMGAHGAFWLFGAICFVGLFFV
IIYVPETQGKTLEDIERKMMGRVRRMSSVANIKPLSFNM.

In some embodiments, the trehalose transporter is a trehalose transporter homolog. In some embodiments, a trehalose transporter homolog comprises at least 70, 75, 80, 85, 90, 95,97, 99 or 100% sequence identity to SEQ ID NO: 5. Each possibility represents a separate embodiment of the invention. In some embodiments, a trehalose transporter homolog comprises at least 85% sequence identity to SEQ ID NO: 5. In some embodiments, a trehalose transporter homolog comprises at least 70, 75, 80, 85, 90, 95, 97, 99 or 100% sequence identity to SEQ ID NO: 6. Each possibility represents a separate embodiment of the invention. In some embodiments, a trehalose transporter homolog comprises at least 85% sequence identity to SEQ ID NO: 6. In some embodiments, a trehalose transporter homolog comprises trehalose import ability. I In some embodiments, a trehalose transporter homolog comprises the ability to actively transport trehalose into a cell. In some embodiments, into a cell is into a cytoplasm of a cell. The functional domains within TRET1 have been well characterized and are known in the art (see for example Kikawada, et al., “Trehalose transporter 1, a facilitated and high-capacity trehalose transporter, allows exogenous trehalose uptake into cells”, Proc. Natl. Acad. Sci. USA, 2007 Jul. 10; 104(28):11585-90, and Kanamori et al., “The trehalose transporter 1 gene sequence is conserved in insects and encodes proteins with different kinetic properties involved in trehalose import into peripheral tissues”, Insect Biochem. Mol. Biol., 2010, January; 40(1):30-7, herein incorporated by reference in their entireties.). Within D. melanogaster TRET1 there are 12 transmembrane domains (amino acids 393-413, 441-461, 474-494, 498-518, 529-549, 553-575, 637-657, 674-694, 701-721, 741-761, 768-788, and 802-822 of SEQ ID NO: 5) which are completely conserved between isoforms 1A and 1B. Conservative mutations in these domains and the retention of hydrophobicity will not alter protein function. Further, as the N-terminus of isoform 1A (amino acids 1-385 of SEQ ID NO: 5) is essentially absent from isoform 1B it is dispensable for transport function. Several conserved amino acids and motifs have been found to be critical to trehalose recognition and transport activity, these include the N-glycosylation stie (N-X-T/s) in the first extracellular loop, the W residue in transmembrane region 10 and QLS motif in transmembrane region 7. Conservative mutations in these regions will still result in a protein having trehalose recognition and transport activity. Most non-conserved mutation in these domains will not result in a protein with trehalase activity. Outside of these domains most mutations will not affect trehalase activity.

In some embodiments, the nucleic acid sequence encodes a trehalase enzyme. In some embodiments, the nucleic acid sequence comprises a coding region that encodes a trehalase enzyme. In some embodiments, the nucleic acid sequence encodes a trehalose transporter. In some embodiments, the nucleic acid sequence comprises a coding region that encodes a trehalose transporter. In some embodiments, the nucleic acid molecule is an expression vector. In some embodiments, the coding region is operatively linked to at least one regulatory element. In some embodiments, the nucleic acid sequence comprises at least one regulatory element operatively linked to the sequence encoding the trehalase. In some embodiments, the nucleic acid sequence comprises at least one regulatory element operatively linked to the sequence encoding the trehalose transporter. In some embodiments, the at least one regulatory element is active in the cell. In some embodiments, the at least one regulatory element is constitutively active. In some embodiments, the at least one regulatory element is inducible. In some embodiments, inducible is inducible in the presence of trehalose. In some embodiments, the at least one regulatory element is a promoter.

The term “operably linked” is intended to mean that the nucleotide sequence of interest is linked to the regulatory element(s) in a manner that allows for expression of the nucleotide sequence (e.g., in a host cell when the vector is introduced into the host cell).

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, expression is protein expression. In some embodiments, expression is cytoplasmic expression. In some embodiments, expression is intracellular expression. In some embodiments, expression is not secretion.

Expressing of a nucleic acid sequence encoding a protein within a cell is well known to one skilled in the art. It can be carried out by, among many methods, transfection, viral infection, or direct alteration of the cell's genome. In some embodiments, the nucleic acid sequence is in an expression vector such as plasmid or viral vector. In some embodiments, the vector is a viral vector. In some embodiments, the virus is a retrovirus. In some embodiments, viral is lentiviral. In some embodiments, the vector is a pCDNA vector. In some embodiments, the pCDNA vector is pCDNA3.1.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter. In some embodiments, the promoter is active in an immune cell. Expression control element specific for immune cells are for example the LCK-promotor which is specific for T cells and the NKP46-promotor which is specific for NK cells and other innate lymphoid cells. Cell specific regulation of expression is well known in the art and any regulatory element or elements that regulate expression such that the protein product is expressed in the cell of the invention may be used.

In some embodiments, the vector is introduced into the cell by standard methods including electroporation (e.g., as described in From et al., Proc. Natl. Acad. Sci. USA 82,5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZcoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 carly promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the trehalase or trehalose transporter), the expression construct of the present invention can also include sequences engineered to optimize stability, production, purification, yield or activity of the expressed polypeptide.

Various methods of editing the genome of the cell to include the heterologous sequence are known in the art and may be employed. These methods include clustered Regularly Interspaced Short Palindromic Repeats (CRISPR)-associated nuclease, Zinc-finger nuclease (ZFNs), meganucleases and transcription activator-like effector nuclease (TALEN) systems of genome editing. In some embodiments, the CRISPR system is a CRISPR-CAS system. In some embodiments, the CAS is CAS9. The use of such systems and the design of targeting constructs are well known in the art and can be used to integrate the heterologous nucleic acid sequence and/or molecule into a genome of a target cell.

By another aspect, there is provided a method of producing a cell of the invention, the method comprising:
a. providing a cell;
b. introducing into the provided cell a nucleic acid sequence encoding for a enzyme capable of converting a non-canonical carbon source into a canonical carbon source to produce an engineered cell;
   thereby producing a cell of the invention.

By another aspect, there is provided a method of producing a cell of the invention, the method comprising:
a. providing a cell;
b. introducing into the provided cell a nucleic acid sequence encoding for a trehalase enzyme to produce an engineered cell;
   thereby producing a cell of the invention.

In some embodiments, the method further comprises introducing into the provided cell a nucleic acid sequence encoding for a non-canonical carbon source transport protein. In some embodiments, the method further comprises introducing into the provided cell a nucleic acid sequence encoding for a trehalose transporter protein. In some embodiments, the introducing is by any method provided hereinabove. In some embodiments, the sequence is a heterologous sequence. In some embodiments, introducing comprises integrating the sequence into the genome of the provided cell. In some embodiments, the method further comprises testing or confirming that the engineered cell is capable of surviving in the presence of trehalose and the absence or depletion of glucose. In some embodiments, the method further comprises selecting a cell that can survive in the presence of trehalose and the absence or depletion of glucose. In some embodiments, the survival is in the absence of glucose. In some embodiments, survival comprises proliferation. Methods of measuring survival are well known in the art and any such method may be used. Methods are also provided hereinbelow. One such method is an MTT assay. In some embodiments, the method is an MTT assay.

### Compositions

By another aspect, there is provided a composition comprising the cell of the invention.

In some embodiments, the composition is a cellular composition. In some embodiments, the composition is a culture composition. In some the composition comprises cell culture media. In some embodiments, the culture media is culture media for the cell type of the cell. In some embodiments, the culture media is immune cell media. In some embodiments, the media is T cell media. In some embodiments, the media is DMEM. In some embodiments, the media is RPMI. In some embodiments, the cell is cultured in the solution. In some embodiments, the solution is media. In some embodiments, the solution is culture media. In some embodiments, the culture media is culture media for the cell. In some embodiments, the media is growth media. In some embodiments, the media is chemically defined media. In some embodiments, the media comprises trehalose. In some embodiments, the media is depleted of glucose. In some embodiments, depleted is devoid of. In some embodiments, depleted comprises reduced levels of glucose as compared to glucose containing media. In some embodiments, reduced is at least 10, 20, 25, 30, 40, 50, 60, 70, 75, 80, 85, 90, 92, 95, 97, 99 or 100% reduced. Each possibility represents a separate embodiment of the invention.

In some embodiments, the composition is a pharmaceutical composition. In some embodiments, composition comprises a pharmaceutically acceptable carrier, excipient or adjuvant. As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions (e.g., the cells) presented herein.

In some embodiments, the composition comprises a therapeutically effective amount of the cells of the invention. The term “therapeutically effective amount” refers to an amount of a composition effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition. In some embodiments, the disease is cancer. In some embodiments, the composition comprises at least 1 million cells of the invention. In some embodiments, the composition consists essentially of the cells of the invention. In some embodiments, the composition consists of the cells of the invention. In some embodiments, the composition is depleted of cells that are not cells of the invention. In some embodiments, the composition is devoid of cells that are not cells of the invention. In some embodiments, the composition is cultured in the absence of glucose and the presence of trehalose for a time sufficient for cells that do not express the heterologous nucleic acid sequence to die. In some embodiments, the cells of the invention make up at least 10, 15, 20, 25, 30, 35, 40, 45, 50, 55, 60, 65, 70, 75, 80, 85, 90, 92, 95, 97, 99 or 100% of the composition. Each possibility represents a separate embodiment of the invention. In some embodiments, the cells of the invention make up at least 40% of the composition. In some embodiments, the cells of the invention make up at least 50% of the composition. In some embodiments, the cells of the invention make up at least 75% of the composition.

In some embodiments, the composition is formulated for administration to a subject. In some embodiments, the subject is a mammal. In some embodiments, the mammal is a human. In some embodiments, the composition is formulated systemic administration. In some embodiments, the composition is formulated administration to a solid cancer. In some embodiments, a solid cancer is a tumor. In some embodiments, the composition is formulated for intratumoral administration. In some embodiments, the composition comprises the cells media. In some embodiments, the media in a chemically defined media. As used herein, the term “chemically defined media” refers to a medium in which all the chemical components are known. In some embodiments, chemically defined media is devoid of animal-based products. In some embodiments, chemically defined media is devoid of animal-based proteins. In some embodiments, the media is protein free media. In some embodiments, the media comprises trehalose. In some embodiments, the media comprises trehalose. In some embodiments, the trehalose is in sufficient concentration as to be a carbohydrate source for the cells. In some embodiments, the trehalose is in sufficient concentration as to be the only carbohydrate source for the cells. In some embodiments, a carbohydrate source is a source for the generation of glucose. In some embodiments, the sufficient concentration is sufficient to keep the cells alive. In some embodiments, the sufficient concentration is sufficient to keep the cells healthy. In some embodiments, alive/healthy is in the absence of or depletion of glucose in the composition. In some embodiments, the media is depleted of glucose. In some embodiments, depleted is devoid of. In some embodiments, depleted comprises reduced levels of glucose. In some embodiments, levels are glucose concentration. In some embodiments, decreased is as compared to glucose containing media. In some embodiments, decreased is as compared to cellular composition comprises cells that cannot metabolize trehalose. In some embodiments, trehalose is extracellular trehalose. In some embodiments, trehalose is intracellular trehalose. In some embodiments, decreased is as compared to cellular composition comprising cells that are not cells of the invention. In some embodiments, reduced is at least 10, 20, 25, 30, 40, 50, 60, 70, 75, 80, 85, 90, 92, 95, 97, 99 or 100% reduced. Each possibility represents a separate embodiment of the invention.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the invention to a patient in need thereof. Other suitable routes of administration can include parenteral, subcutaneous, oral, intramuscular, or intraperitoneal.

In some embodiments, the cells of the invention are for use in a method of the invention. In some embodiments, the compositions of the invention are for use in a method of the invention. In some embodiments, the method is a method of adoptive cell therapy. In some embodiments, the method is a method of treating a subject in need thereof. In some embodiments, the method is a method of treating cancer. In some embodiments, the cancer is in a subject.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

### Methods of Use

By another aspect, there is provided a method for adoptive cell therapy in a subject, the method comprising administering to the subject a pharmaceutical composition of the invention, thereby performing adoptive cell therapy.

By another aspect, there is provided a method of treating cancer the method comprising contacting the cancer cells with the cells of the invention, thereby treating cancer.

In the context of the present invention the “adoptive cell therapy” is a general term not restricted only to T cells therapy and not restricted only to CAR-T therapy. This term in the context of the invention refers to any anti-cancer therapy wherein cells of the immune system are obtained ex vivo, manipulated ex vivo in order to improve their anti-cancer properties and introduced to the patient in a single or multiple doses. In some embodiments, the cells are obtained from the subject and re-introduced back to the subject. In some embodiments, the cells are allogeneic to the subject. In some embodiments, the cells are syngeneic to the subject. In some embodiments, the cells are autologous to the subject.

In some embodiments, the subject is a subject in need of the therapy. In some embodiments, the subject is a mammal. In some embodiments, the subject is a human. In some embodiments, the subject suffers from a disease treatable by the therapy. In some embodiments, the subject is in need to the method of the invention. In some embodiments, the subject suffers from a disease. In some embodiments, the disease is treatable by adoptive cell transfer. In some embodiments, the disease is treatable by transfer of the cells of the invention. In some embodiments, the disease is treatable by transfer of cells of the same cell type as the cells of the invention. In some embodiments, the disease is characterized by regions of low or depleted glucose.

In some embodiments, the disease is cancer. In some embodiments, the method is a method of treating cancer. In some embodiments, the cancer is a solid cancer. In some embodiments, the cancer is a tumor. As used herein “cancer” is a disease associated with dysregulated cell proliferation. In some embodiments, the cancer is selected from hepato-biliary cancer, cervical cancer, urogenital cancer (e.g., urothelial cancer), testicular cancer, prostate cancer, thyroid cancer, ovarian cancer, nervous system cancer, ocular cancer, lung cancer, soft tissue cancer, bone cancer, pancreatic cancer, bladder cancer, skin cancer (e.g., melanoma), intestinal cancer, hepatic cancer, rectal cancer, colorectal cancer, esophageal cancer, gastric cancer, gastroesophageal cancer, breast cancer (e.g., triple negative breast cancer), renal cancer (e.g., renal carcinoma), skin cancer, head and neck cancer, leukemia and lymphoma. In some embodiments, the cancer is skin cancer. In some embodiments, the skin cancer is melanoma.

In some embodiments, the cancer comprises a region of reduced glucose levels. In some embodiments, the tumor microenvironment (TME) comprises reduced glucose levels. In some embodiments, the region is the TME. In some embodiments, glucose levels are glucose concentration. In some embodiments, reduced is as compared to a healthy control. In some embodiments, reduced is as compared to a non-cancerous region in the subject. In some embodiments, the non-cancerous region is in the same tissue or location as the cancer. In some embodiments, non-cancerous is non-tumor. In some embodiments, the contacting is contacting with the cancer cells. In some embodiments, the contacting is with the tumor. In some embodiments, the contacting is with the TME. In some embodiments, the contacting is with the region.

In some embodiments, the treating comprises contacting with the cells. In some embodiments, the treating comprises contacting with the composition of the invention. In some embodiments, the contacting comprises administering the cells of the invention. In some embodiments, the treating comprises administering the composition of the invention.

In some embodiments, the method further comprises confirming the cancer comprises a region comprising a glucose concentration below a predetermined threshold. In some embodiments, the method comprises measuring glucose concentration in the cancer. In some embodiments, in the cancer is in a region of the cancer. In some embodiments, the region is the TME. In some embodiments, the TME is a region around the cancer. In some embodiments, the TME is a region adjacent to the cancer. In some embodiments, the TME is a region proximal to the cancer. In some embodiments, proximal is within the same tissue as the cancer. In some embodiments, the confirming or measuring is in the subject. In some embodiments, the confirming or measuring is in the cancer of the subject. In some embodiments, the confirming or measuring is receiving confirmation or measurements. In some embodiments, the confirming or measuring is before the administration or contacting. In some embodiments, a measurement below the predetermined threshold indicates the subject is suitable for the method of the invention. In some embodiments, a measurement at or above the predetermined threshold indicates the subject is not suitable for the method of the invention. In some embodiments, after the measuring the method is continued. In some embodiments, after the measuring the method is discontinued. In some embodiments, an alternative therapy is administered.

In some embodiments, the predetermined threshold is the glucose concentration in a healthy control. In some embodiments, the control is the same tissue or region comprising the cancer. In some embodiments, the predetermined threshold is the glucose concentration in the organ or region in which the cancer is found but in a healthy subject. In some embodiments, the region is a region in the body. In some embodiments, the predetermined threshold is the glucose concentration in a non-cancerous location in an organ or bodily region in the subject. In some embodiments, the organ or bodily region is the same as the one that comprises the cancer.

In some embodiments, below a predetermined threshold is reduced. In some embodiments, below is significantly below. In some embodiments, below is below by at least 5, 10, 15, 20, 25, 30, 35, 40, 45, 50, 55, 60, 65, 70, 80, 85, 90, 92, 95, 97, 99 or 100% below. Each possibility represents a separate embodiment of the invention.

In some embodiments, the method further comprises receiving cells from the subject. In some embodiments, the cells are obtained from the subject. In some embodiments, the method comprises extracting cells from the subject. In some embodiments, the cells are immune cells. In some embodiments, the cells are from a sample. In some embodiments, the sample is from, obtained from or extracted from the subject. In some embodiments, the sample is a peripheral blood sample. In some embodiments, the sample is a bone marrow sample. In some embodiments, the cells are peripheral blood mononuclear cells (PBMCs). In some embodiments, the method further comprises providing cells. In some embodiments, the cells are primary cells. In some embodiments, the cells are cultured ex vivo. In some embodiments, the cells are expanded. In some embodiments, the cells are activated. In some embodiments, the cells are engineered to express the heterologous nucleic acid sequence that encodes a trehalase enzyme. In some embodiments, engineered is genetically engineered. In some embodiments, cells are engineered to express the heterologous nucleic acid sequence that encodes a trehalose transporter. In some embodiments, the engineering comprises contacting the cells with the nucleic acid molecule. In some embodiments, contacting is transducing. In some embodiments, contacting is transfecting. In some embodiments, the engineering comprises infecting the cells with a virus comprising the heterologous nucleic acid sequence. In some embodiments, the virus integrates the heterologous sequence into the genome of the cell. In some embodiments, the engineering comprises directly editing the genome of the cell. In some embodiments, the editing is with a genome editing protein or complex. In some embodiments, the method further comprises returning the engineered cells to the subject.

By another aspect there is provided an in vivo aspect of the present invention wherein the method comprises: administering to a subject in need of such treatment an expression vector encoding the heterologous nucleic acid sequence of the invention. A person with skill in the art will appreciate that a gene can also be expressed from a nucleic acid construct administered to the individual employing any suitable mode of administration, described hereinabove (i.e., in vivo gene therapy).

For the in vivo aspect the vector for expression comprises the nucleic acid sequences required to encode for trehalase and optionally further comprising a nucleic acid sequence that encode a trehalose transporter, the sequences being present under suitable control elements for the selective expression in immune cells. In some embodiments, the sequences are operably linked to an immune cell specific regulatory element. In some embodiments, the vectors are delivered via targeted delivery to immune cells. Methods are target delivery of nucleic acids, such as by lipid nanoparticles (LNPs) are known in the art and can be employed for the in vivo method of the invention.

In the vivo aspect the selective targeting into immune cells can be done by constructing a delivery vehicle carrying on its surface entities enabling selective binding and internalization into immune cells, by immune-cell specific receptors. Non limiting examples of such receptors include CD8 on T cells and CD56 on NK cells.

In some embodiments, the method further comprises contacting the cancer with the non-canonical carbon source. In some embodiments, the method further comprises contacting the cancer with trehalose. In some embodiments, the contacting comprises administering the non-canonical carbon source to the subject. In some embodiments, the contacting comprises administering trehalose to the subject. In some embodiments, the administering trehalose is before the administering of the cells of the invention. In some embodiments, the administering trehalose is concomitant to the administering of the cells of the invention. In some embodiments, the administering trehalose is after the administering of the cells of the invention. In some embodiments, after is at least 1, 2, 3, 4, 5, 6 or 7 days after. Each possibility represents a separate embodiment of the invention. In some embodiments, after is not more than 1, 2, 3, 4, 5, 6, or 7 days after. Each possibility represents a separate embodiment of the invention. In some embodiments, after is 1-3 days after. In some embodiments, after is 1-2 days after. In some embodiments, the administering is administering a trehalose composition of the invention. In some embodiments, the composition of the invention is for use in combination with a composition comprising trehalose. In some embodiments, the administering is administering a trehalose composition of the invention. In some embodiments, a composition comprising trehalose is a trehalose composition of the invention.

In some embodiments, the trehalose administration is continued for the as long as the adoptive cell therapy continues. In some embodiments, the trehalose administration is continued for as long as cells of the invention survive in the subject. In some embodiments, the trehalose administration is continued until treatment of the subject is completed. In some embodiments, treatment is cancer treatment. In some embodiments, treatment is a method of the invention. In some embodiments, the trehalose is repetitively administered every 2-5 days. In some embodiments, the trehalose is repetitively administered every other day. In some embodiments, the trehalose is repetitively administered every 1, 2, 3, 4, 5, 6, or 7 days. Each possibility represents a separate embodiment of the invention. In some embodiments, the trehalose is administered for 2 to 10 weeks. In some embodiments, the trehalose is administered for at least 1, 2, 3, 4, 5, 6, 7, 8, 9 or 10 weeks. Each possibility represents a separate embodiment of the invention.

The mode of administration of the cells and the trehalose need not be the same. A skilled artisan will understand that for example, while the cells are introduced via IV the trehalose may be administered by a different mode using another carrier, such as orally. However, as some of the trehalose sources are not transferred through the gut, or are degraded in the gut, IV administration of the trehalose may be performed. In some embodiments, the administration is as described herein. In some embodiments, the administration is IV administration. In some embodiments, the administration is intratumoral administration.

### Trehalose Compositions and Kits

By another aspect, there is provided, a pharmaceutical composition comprising the non-canonical carbon source in pharmaceutical grade purity together with a pharmaceutically acceptable carrier.

By another aspect, there is provided, a pharmaceutical composition comprising trehalose in pharmaceutical grade purity together with a pharmaceutically acceptable carrier.

The above pharmaceutical composition is for the administration in during adoptive cell transfer therapy together the with the above genetically engineered cells engineered with heterologous nucleic acid sequences that encode at least one heterologous protein required for metabolizing a non-canonical carbon source. In some embodiments, the composition is for use in a method of the invention. In some embodiments, the composition is formulated for systemic administration. In some embodiments, the composition is formulated for intratumoral administration. In some embodiments, the composition is formulated for administration to the subject. In some embodiments, the composition further comprises the cells of the invention. In some embodiments, the purity is sufficient to administer to a human subject.

In some embodiments, the pharmaceutical composition comprises a high dose of trehalose. In some embodiments, a high dose is a dose higher than is used when treating a disease other than cancer. In some embodiments, the disease other than cancer is a neurodegenerative disease. In some embodiments, the neurodegenerative discase is characterized by misfolded proteins, protein aggregation or both. In some embodiments, the neurodegenerative disease is Parkinson's disease. In some embodiments, the neurodegenerative disease is selected from Parkinson's disease, Alzheimer's disease, amyotrophic lateral sclerosis (ALS), Huntington's disease and spinocerebellar ataxia (SCA). In some embodiments, the disease other than cancer is an infection. In some embodiments, the infection is a viral infection. In some embodiments, the virus is a corona virus. In some embodiments, the corona virus is SARS-COV-2.

By another aspect, there is provided a kit comprising a composition of the invention.

In some embodiments, the kit comprises the cells of the invention. In some embodiments, the kit comprises the cellular composition of the invention and the trehalose composition of the invention. In some embodiments, the kit comprises instructing stating the cellular composition is for use in combination with a trehalose composition. In some embodiments, the kit comprises instructing stating the trehalose composition is for use in combination with a cellular composition. In some embodiments, the cellular composition is a cellular composition of the invention.

### Methods of Determining Patient Suitability

By another aspect, there is provided a method of determining suitability of a subject to be treated by a method of the invention, the method comprising measuring glucose levels in a cancer of the subject, wherein glucose below a predetermined threshold indicates the subject is suitable to treatment, thereby determining suitability of a subject.

In some embodiments, glucose levels at or above the predetermined threshold indicate the subject is not suitable for treatment. In some embodiments, the method further comprises performing a method of the invention on a subject suitable subject. In some embodiments, a suitable subject is a subject determined to be suitable. In some embodiments, the method further comprises administering an alternative therapy to a subject found not to be suitable. In some embodiments, the alternative therapy is adoptive cell transfer with cells that have not been modified by a method of the invention.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Cloning and Expression of Drosophila Treh and Tret1

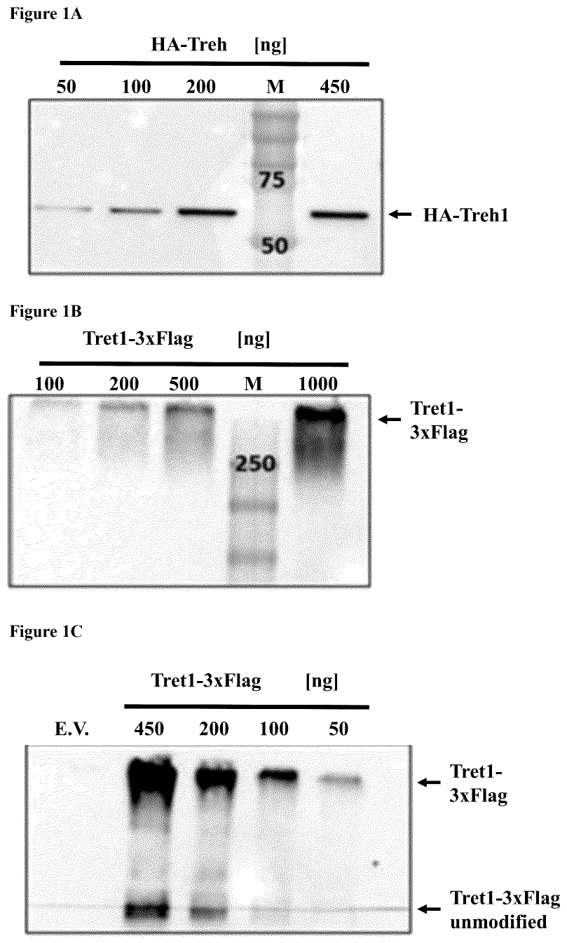

Insects' cells are capable of utilizing trehalose as a carbon source to support cellular metabolism. This process requires the membrane trehalose transporter, Tret1, and the intra-cellular trehalase, Treh which catalyzes the conversion of trehalose to glucose inside the cell cytoplasm. To clone the Drosophila Treh gene into a canonical expression vector, the Treh gene was amplified using specific primers from a Drosophila melanogaster cDNA library to generate HA-tagged Treh. The primers used were as follows: Forward primer: GCGAAGCTTGCCACCATGTACCCATACGATGTTCCAGATTACGCTGCCTCTCCA GCGAATCC (SEQ ID NO: 9); and Reverse primer: CAGAATTCCTACTGCCGCTTTTCACCTGCTAG (SEQ ID NO: 10). Then, the PCR product was cloned into a pCDNA3.1 mammalian expression vector. Tret1-3×Flag pCDNA3.1 expression plasmid, encoding TRET1 isoform A (Tret1-1A), was purchased from NovoPro (novoprolabs.com). Next to test the ability of human cells to express Treh and Tret1genes from Drosophila melanogaster, HEK-293T cells were transiently transfected with either of the plasmids at various concentrations. Then, protein extracts from the cells were subjected to immunoblot analysis using anti-Flag or anti-HA to detect expression of Tret1-3×Flag or HA-Treh respectively. The overexpression of the HA-Treh gave rise to a band corresponding to the expected molecular weight, 62.6 kDa (FIG. 1A). However, overexpression of the Tret1-3×Flag gave rise to a band corresponding to >300 kDa, which is much higher than the expected, 97 kDa (FIG. 1B). Treatment of the cells with the secretion inhibitor, brefeldin A, led to the appearance of a band corresponding to 97 kDa (FIG. 1C). These results suggest that, as expected, Tret1 being a membrane-localized transporter, undergoes posttranslational modifications, most likely, glycosylation at the Golgi. Therefore, it was concluded that TRET1 is modified and likely to be transported to the plasma membrane. Co-transfection of both constructs gave rise to the two expected bands (FIG. 1D).

### Example 2: Treh and Tret1 Co-Expression Rescues the Proliferation and Survival of HEK-293T Cells in Glucose-Free, Trehalose Supplemented Medium

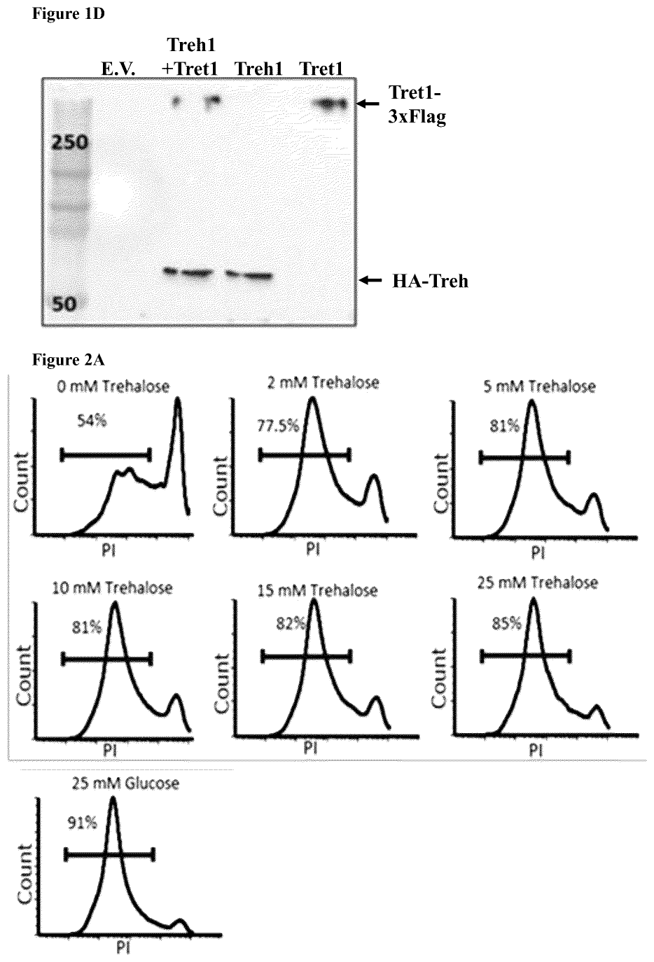

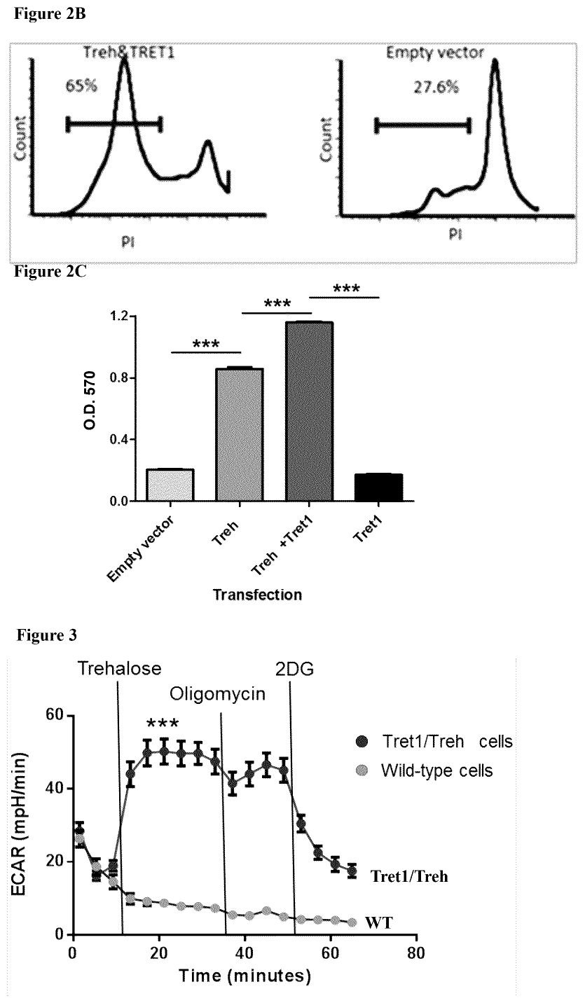

To test the function of delivered Treh and Tret1 genes in HEK-293T cells the survival of Treh/Tret1 expressing cells was tested in the absence or presence of various trehalose concentrations in glucose-free media. A substantial rescue was observed already at 2 mM trehalose with increased survival of the cells in correlation with the trehalose concentration (FIG. 2A). Notably, cells grown in 25 mM trehalose showed a similar survival rate to cells grown in 25 mM glucose. In addition, Trehalose could rescue the survival of Treh/Tret1 expressing cells but not of empty vector transfected cells (FIG. 2B). Finally, using MTT assay it was demonstrated that Treh/Tret1 co-expression rescues both the viability and proliferation of the HEK-293T cells in glucose-free media supplemented with trehalose (FIG. 2C). Notably, Treh-transfected cells demonstrated significantly higher MTT staining in comparison to empty-vector or Tret1-transfected cells (FIG. 2C). These results indicate that human cells have endogenous Trehalose transporter activity, very likely due to glucose transporter SLC2A8 (also called GLUT8). Nevertheless, this endogenous Trehalose transporter seems inferior to the insect Tret1. Overall, the results indicate that canonical cells expressing insect Treh and Tret1 genes enable the utilization of trehalose as an alternative carbon source to glucose.

### Example 3: Treh and Tret1 Co-Expression Allow Utilization of Trehalose as a Carbon Source for Glycolysis

These results clearly demonstrate that expression of insect's Treh and Tret1 genes induce survival and proliferation of mammalian cells in a trehalose dependent manner. These results suggest that Treh and Tret1 expression allows mammalian cells to utilize trehalose as an alternative carbon source to support glucose-based metabolism. To directly examine this notion, whether trehalose restores glycolysis of Treh/Tret1 expressing cells was tested. For this purpose, the extracellular acidification rate (ECAR), as a proxy for lactate secretion, of Treh/Tret1 or wild-type cells, was analyzed using Seahorse analysis. HEK293T cells expressing Treh/Tret1, but not wild-type cells, showed a significant increase in their ECAR following the addition of trehalose (FIG. 3). This elevated ECAR was significantly decreased following the addition of the glycolysis inhibitor, 2-Deoxy-D-glucose (2-DG) (FIG. 3). Notably, the addition of oligomycin, a mitochondrial ATP synthase inhibitor, did not lead to an elevation in ECAR (FIG. 3), suggesting that trehalose led to the maximal glycolytic capacity of the cells. These results strongly indicate that insect Treh and Tret1 support the survival and proliferation of mammalian cells in a glucose-free medium by utilizing trehalose as a carbon source for glycolysis.

### Example 4: Treh and Tret1 Co-Expression Allow the Long-Term Survival and Proliferation of Human T Cell Line

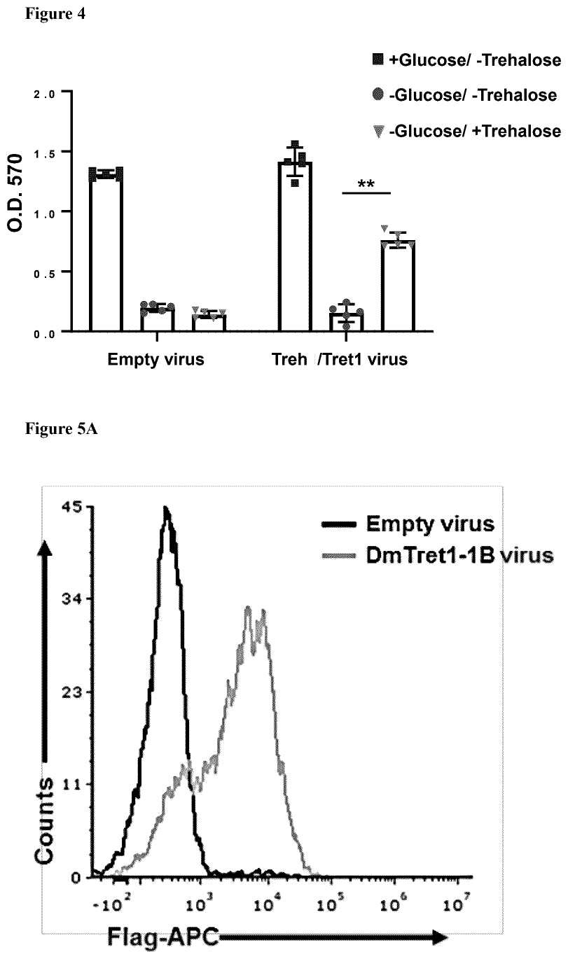

Finally, to test whether the insect proteins have the potential to make T cells “metabolically superior”, the function of Treh and Tret1 genes after delivery into the human T cell line Jurkat was tested. To this aim, Jurkat cells were transduced with retroviruses expressing both Tret1-3×Flag and Treh-GFP, or GFP alone. Cells were then sorted by FACS for GFP positive cells and were grown for a week in a glucose-free medium supplemented with 10 mM Glucose (Blue square), 5 mM trehalose (Green triangle) or neither (Red circle). Cell-survival and proliferation were then analyzed by MTT assay. The results clearly demonstrate that Jurkat cells co-expressing Treh/Tret1 survive and proliferate in the presence of both glucose and trehalose. However, Jurkat cells that do not express Treh/Tret1 can survive only in the presence of glucose (FIG. 4). These results show that co-expression of Treh/Tret1 provides Jurkat cells with the means to utilize trehalose instead of glucose and therefore highlight the ability of this approach to make T cells glucose-independent, and therefore more resilient to glucose-poor tumor microenvironments.

### Example 5: Use of Tret1 Short Variant

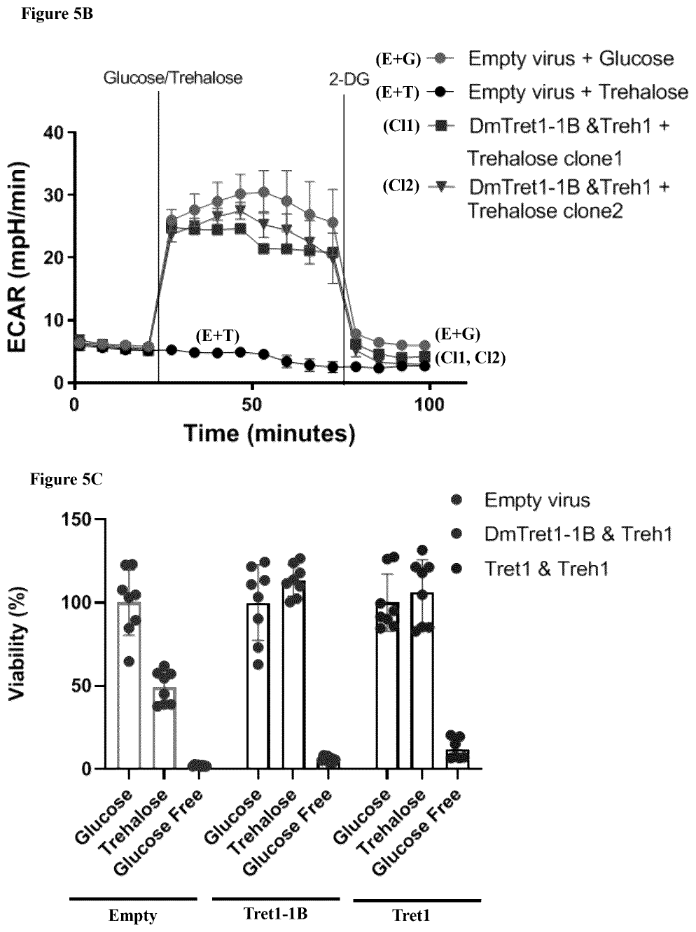

In an effort to improve infection efficiency, a short isoform of drosophila Tret1 (Tret-1B) was inserted into the same lentiviral backbone as the longer isoform and tested. First, the expression of Tret1-1B was validated by flow cytometry using intracellular staining for the Flag tag (FIG. 5A). Next, Tret1-1B/Treh expressing Jurkat cells were generated and tested for improved metabolism and function under glucose-restricted conditions. To this end it was tested whether Tret1-1B/Treh expressing Jurkat cells can utilize trehalose for aerobic glycolysis by testing their extra cellular acidification rate (ECAR) as a proxy for lactate secretion using Seahorse analysis as before. The results clearly demonstrate that Tret1-1B/Treh expressing Jurkat cells increased their ECAR following trehalose injection while cells that do not express these genes failed to do so (FIG. 5B). Injection of glucose led to an increase in ECAR in all cells regardless of Treh/Treh expression. Next, to test improved function under glucose-restricted conditions it was assessed whether Tret1-1B/Treh expression provides the cells with survival and proliferation advantages. For this, Tret1-1B/Treh-expressing or control Jurkat cells were grown in 3 different media, glucose-containing medium, glucose-free and trehalose-supplemented medium, or glucose-free and trehalose-free medium. The results show that only T cells expressing both Tret1-1B (or Tret1-1A) and Treh were able to grow in trehalose containing medium (FIG. 5C). Next, whether the constructs were functional in other immune cells was tested. The human NK cell line, NK92, was used as it is currently in broad use for ACT therapy. NK92 cells were transduced with both lentiviruses and selected for the Tret1-1B/Treh expressing cells by culturing them in a glucose-free, trehalose-supplemented medium. As a control, half of the cells were cultured in glucose-containing medium. The cells were then analyzed by flow cytometry for the relative abundance of Tret1-1B-GFP/Treh-RFP double positive cells at various time points (FIG. 5D). The results show that the presence of GFP/RFP double positive cells increased with time in the cells that were grown on trehalose, while these cells were barely detectable when grown in glucose-containing medium (FIG. 5D). This shows that in NK cells as well the double expression of Tret1 and Treh provides a survival advantage in the absence of glucose. Overall, these results demonstrate that expression of Treh and Tret1 provide both T and NK cells with the means to uptake trehalose and catalyze its breakdown into glucose.

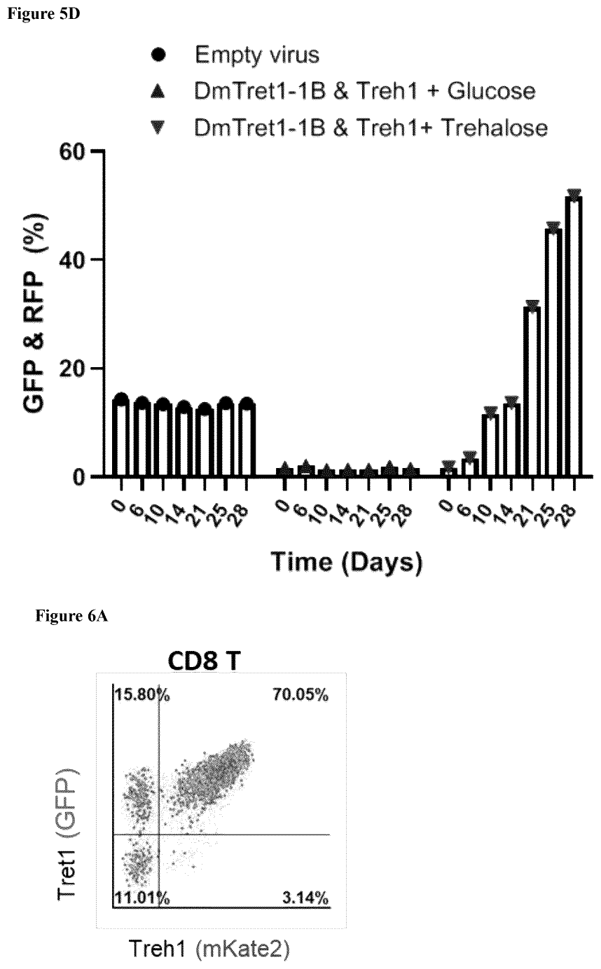

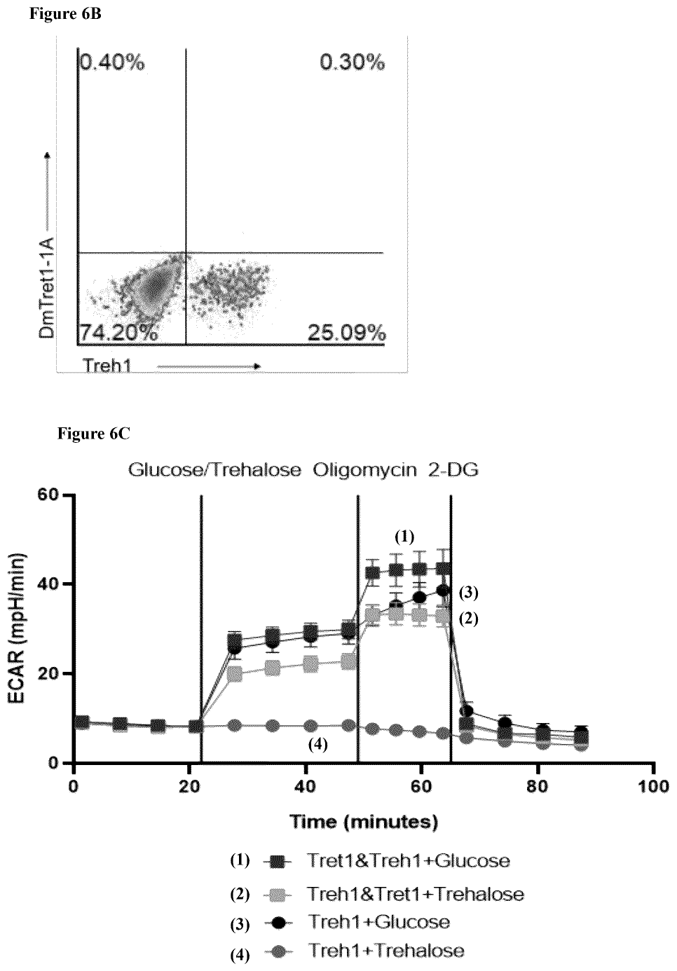

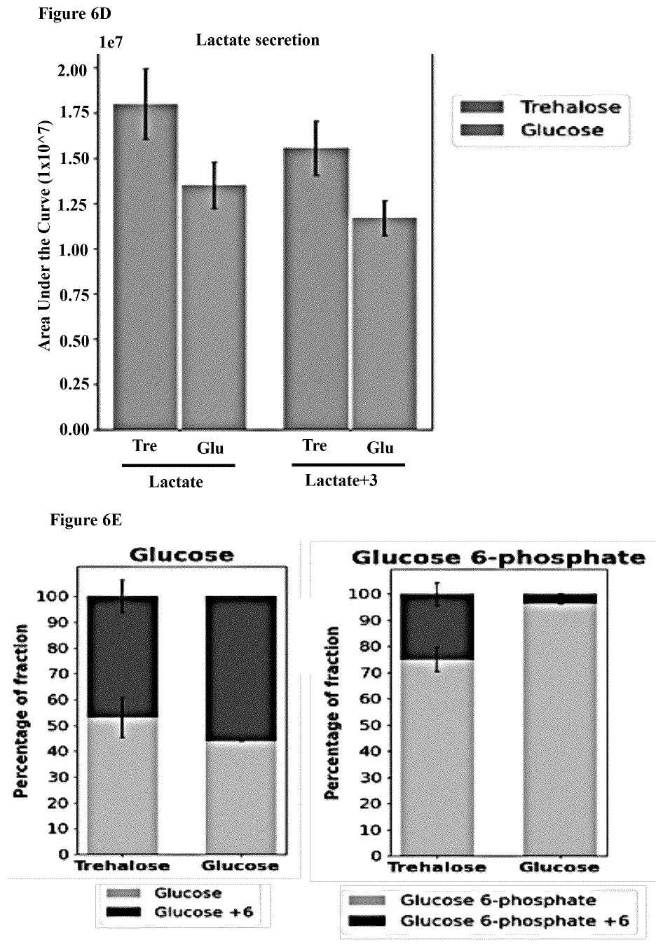

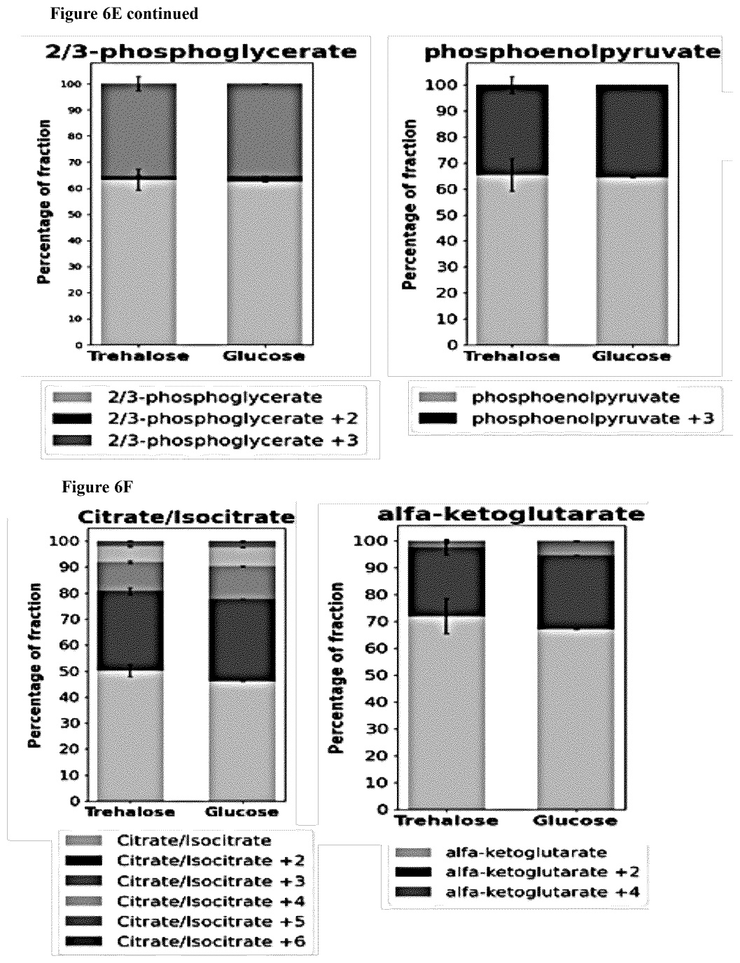

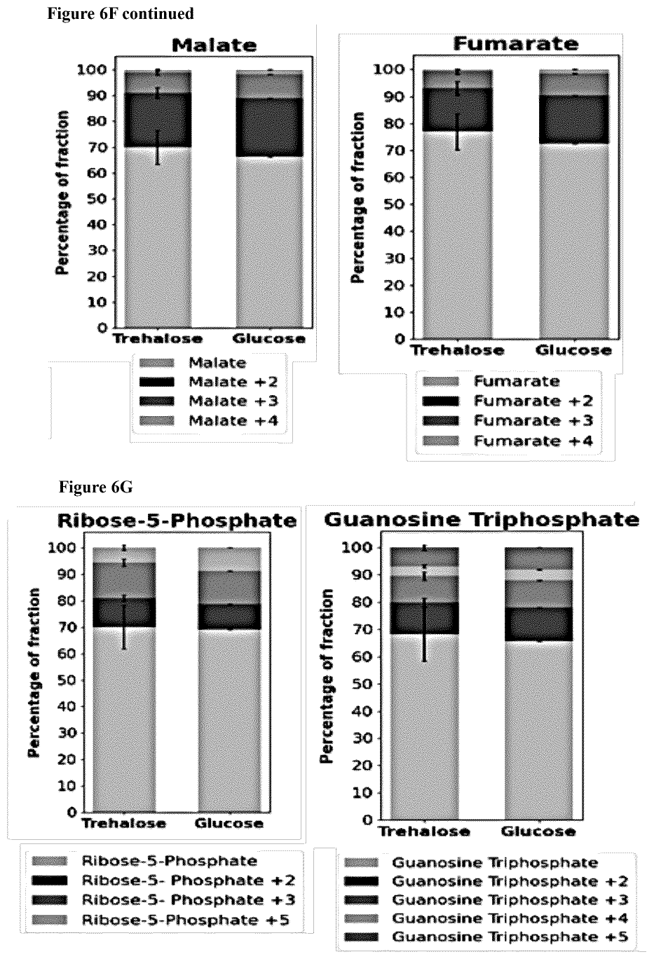

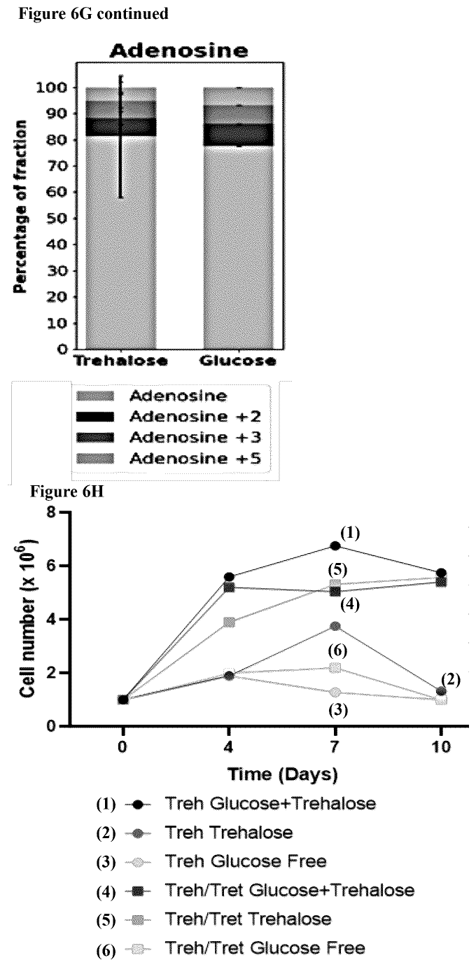

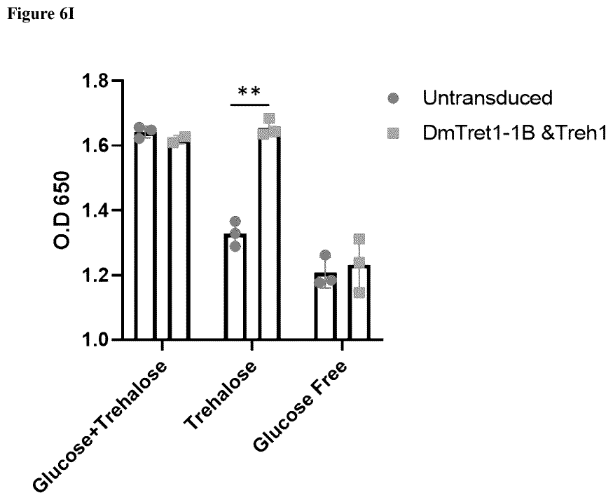

Next, Treh1 and DM-Tret1-1B were cloned into the MSVG retrovirus vector and then these vectors were transduced into activated human T cells derived from PBMCs (PBMCs were activated using anti-CD3 and anti-CD28) (FIG. 6A). Highly efficient transduction was achieved with the short Tret1. In contrast, the longer isoform transduced poorly (FIG. 6B).

Human T cells expressing both Treh1 and Dm-Tret1-1B were able to utilize trehalose for glycolysis, whereas T cells expressing only Treh1 were unable to do so (FIG. 6C). Additionally, metabolic isotope tracing analysis was used to compare the utilization of 13C6-glucose and 13C12-trehalose by the T cells. It was found that trehalose integrates into the same metabolic pathways as glucose, including glycolysis (FIG. 2D-E), the pentose phosphate pathway (FIG. 2F), and the TCA cycle (FIG. 2G). Furthermore, T cells expressing Treh1 and Dm-Tret1-1B exhibited proliferation in the presence of trehalose (FIG. 2H). Notably, the intensity of interferon-γ (INF-γ) cytokine release upon CD3 stimulation was preserved in trehalose media as compared to glucose media, while secretion was significantly reduced in glucose-free media (FIG. 2I). Taken together these findings demonstrate that co-expression of Treh1 and Tret1-1B enables immune cells (both cell lines and primary cells) to utilize trehalose as an alternative carbon source. Moreover, the simultaneous expression of these genes is sufficient to enable glucose-dependent human T cells to survive, proliferate and produce a cytotoxic response in a glucose free environment supplemented with trehalose.

### Example 6: Insertion of Heterologous Treh/Tret1 Genes to Improve the Metabolism and Function of Glucose-Restricted CTLs

Transgenic mice expressing both Tret1-1B and Treh are generated. T cells from these mice are used as donor cells for testing adoptive cell therapy in melanoma-bearing mice as described hereinbelow.

Treh/Tret1-transduced human and mouse CTLs or CTLs from transgenic mice are grown in medium containing various glucose concentrations, (0, 2, 5 and 25 mM) either supplemented or not with 25 mM trehalose. Effector functions of the cells are measured by flow cytometric analysis for IFNγ intracellular staining as an indicator for cytokine secretion activity, and cell trace dilution as an indicator of cell proliferation.

To evaluate the metabolic fitness of the cells their extracellular acidification rate (ECAR) is measured by seahorse analysis as an indicator for glycolysis rate. In addition, the phosphorylation level of AKT, AMPK, and S6 are measured as markers for the activity of the mTOR pathway.

Next, the metabolic fitness of the cells is further tested in an experimental setting where the CTLs compete with tumor cells for glucose availability. For this purpose, the effector functions and metabolic fitness (as mentioned above) of Treh/Tret1-transduced WT mouse CTLs that are grown in the presence of tumor cells with high and low glycolysis rate (EL4-OVA and B16-melanoma cell lines) are measured. Highly glycolytic tumor cells are generated by pretreatment with the Akt activator 4-hydroxytamoxifen (4-HT) or by overexpression of the glucose transporter, Glut1. Poorly glycolytic tumor cells are generated by pretreatment with the inhibitor of mouse target of rapamycin (mTOR). Finally, the effect of trehalose addition on the antigen-specific cytotoxic activity of Treh/Tret1-transduced CTLs is tested. For this, as a source for the Treh/Tret1-transduced CTLs CD8 T cells from two TCR-transgenic mice, the OVA-specific, OT-I, and gp100 specific, Pmel, are used. The EL4-OVA cell line is used as a target cell for OT-I CTLs. The B16-F10/mhgp100/Db (B16-F10 melanoma cells double transfected with mouse-human gp100 and the H-2 Db allele) cell line is used as target cells for Pmel CTLs. Treh/Tret1-transduced CTLs from each of the transgenic mice are incubated for 5 hours with highly or poorly glycolytic corresponding tumor cells. Then, cells are collected, and apoptosis of the tumor cells is measured by Annexin V staining using flow cytometry. Results from these experiments demonstrate that Treh/Tret1 expression leads to a recovery in metabolism, effector functions and cytotoxic capability of the T cells and that this effect is more significant in glucose-restricted conditions in comparison to glucose abundant conditions. Similar experiments are performed with human primary T cells or PBMCs converted to T cells. NK cells are also examined in a similar manner.

### Example 7: Additive Efficacy of TILs Engineered to Metabolize Trehalose for ACT

Tumor-imposed nutrient restrictions can lead to T cell hypo-responsiveness even when tumors are highly antigenic. Moreover, it has been shown that this nutrient competition between tumors and T cells can dictate cancer progression. Therefore, it is highly important to provide CTLs with the ability to utilize trehalose instead of glucose as this not only provides them with the means to overcome glucose-restricted regions, but it can also practically enhance CTLs activity and has an impact on tumor progression.

Therefore, it is demonstrated that CTLs that are engineered to metabolize trehalose overcome hypo-responsiveness imposed by solid tumor microenvironment induced glucose restriction and that this is translated to inhibition in tumor progression.

Two tumor models are used to test in vivo efficacy of the Treh/Tret1-expressing CTLs. In the first model male CD1nu/nu mice are injected subcutaneously in the upper back with 1×10\hat{ }6 624 mel cells admixed 1:1 with Matrigel (BD Biosciences). Five days later, mice are injected in the tumor area with 1×10\hat{ }6 Treh/Tret1-expressing CD8+ or WT CD8+ lymphocytes obtained after co-incubation of gp100154-162-reactive T cells (T cells reactive to the peptide KTWGQYWQV (SEQ ID NO 11). Following T-cell transfer, mice are injected with trehalose (2 grams per kg body weight) into the tumor site daily for 5 days. Tumor progression is determined by measuring tumor size over time. Treh/Tret 1-expressing cells are superior to WT cells at slowing cancer progression.

The second model is a well-known model for melanoma in which the B16-F10/mhgp100/Db melanoma cell line is injected subcutaneously to mice. The source for the CTLs is in-vitro activated CD8 T cells from Pmel-1 transgenic mice transduced with bicistronic retrovirus expressing Treh and Tret1. C57BL/6 mice are anesthetized. B16-F10/mhgp100/Db mouse-melanoma cells (1×106) are injected subcutaneously (0.1 ml total volume) in the back of mice. Six days later, the tumor-bearing mice are subjected to an irradiation dose of 500 rad. On day 7, Treh/Tret expressing gp100-specific pmel-1-GFP CTLs are adoptively transferred intravenously (0.1 ml total volume) to the tail vein. From day 8 onwards mice are administered intraperitoneally twice a day for 5 days with 50 μl (2 g/kg) of trehalose or vehicle. As a control, CTLs that do not express Treh and Tret are also administered. One group of mice serves to assess tumor progression; this is achieved by measuring tumor size with respect to time. When tumor size reaches 15 mm in one of the measured dimensions or becomes necrotic, and the mouse is sacrificed. The second group of mice serves for measurement of TIL's metabolic fitness and effector functions (as described hereinabove) and for whole tumor analysis, such as immunohistochemistry.

Higher percentages of metabolically and functionally recovered CTLs, as well as reduced tumor size is observed in mice that receive Treh/Tret1 expressing CTLs in comparison to normal CTLs. This strongly indicates that metabolic reprogramming of T cell to utilize trehalose as a carbon source can bypass glucose-deprivation induced suppression imposed by solid tumors.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A mammalian immune cell comprising a heterologous nucleic acid sequence that encodes for a trehalase enzyme and a heterologous nucleic acid sequence that encodes a trehalose transporter protein.

2. The cell of claim 1, wherein at least one of:
a. said mammal is a human;
b. said immune cell is selected from a CD8 T cell, an NK cell, a tumor infiltrating lymphocyte (TIL), a macrophage, a dendritic cell, and a chimeric antigen receptor (CAR) expressing or recombinant T cell receptor expressing immune cell; and
c. said cell has been extracted from a subject, grown in culture and made to express said heterologous nucleic acid sequences.

3. (canceled)

4. The cell of claim 1, wherein said trehalase enzyme is an insect trehalase, said trehalose transporter protein is an insect trehalose transporter protein or both.

5. The cell of claim 4, wherein said insect trehalase enzyme is drosophila melanogaster trehalase (TREH) or a functional fragment, variant, or homolog thereof.

6. The cell of claim 5, wherein said TREH comprises or consists of an amino acid sequence selected from SEQ ID NO: 1-3 or a fragment, variant, or homolog thereof comprising at least 85% sequence identity and comprising trehalase enzymatic function.

7. The cell of claim 6, wherein said TREH comprises or consists of SEQ ID NO: 1 or a fragment, variant or homolog thereof comprising at least 85% sequence identity and comprising trehalase enzymatic function.

8. The cell of claim 4, wherein said insect trehalose transporter protein is drosophila melanogaster trehalose transporter 1 (TRET1) or a functional fragment, variant or homolog thereof.

9. The cell of claim 8, wherein said TRET1 comprises or consists of an amino acid sequence selected from SEQ ID NO: 5-6 or a fragment, variant, or homolog thereof comprising at least 85% sequence identity and comprising trehalose transport activity.

10. The cell of claim 9, wherein said TRET1 comprises or consists of SEQ ID NO: 6 or a fragment, variant, or homolog thereof comprising at least 85% sequence identity and comprising trehalose transport activity.

11. The cell of claim 1, wherein said heterologous nucleic acid sequence encoding a trehalase enzyme, said heterologous nucleic acid sequence encoding a trehalose transporter protein or both is operatively linked to at least one regulatory element active in said cell, optionally wherein said regulatory element is constitutively active in said cell or is inducible to activity in said cell.

12. (canceled)

13. (canceled)

14. A pharmaceutical composition comprising a cell of claim 1 and a pharmaceutically acceptable carrier, excipient or adjuvant, optionally wherein said pharmaceutical composition is formulated for administration to a subject, formulated for systemic administration, formulated for administration to a tumor or any combination thereof.

15. (canceled)

16. The pharmaceutical composition of claim 14, further comprising trehalose.

17. (canceled)

18. A method for adoptive cell therapy in a subject in need thereof, the method comprising administering to said subject a pharmaceutical composition of claim 14 and administering trehalose to said subject, thereby performing adoptive cell therapy.

19. The method of claim 18, wherein said subject suffers from a solid cancer, optionally wherein said solid cancer comprises a tumor microenvironment (TME) comprising reduced glucose concentration as compared to non-tumor regions within said subject.

20. (canceled)

21. The method of claim 19, further comprising before said administering confirming in said subject that a TME of said solid cancer comprises a glucose concentration below a predetermined threshold, and wherein said predetermined threshold is the glucose concentration in the organ in which said solid cancer is found in a control subject or a non-cancerous location in said organ in said subject.

22. (canceled)

23. The method of claim 18, further comprising receiving immune cells obtained from said subject, ex vivo genetically engineering said cells to express said heterologous nucleic acid sequences that encodes for a trehalase enzyme and said heterologous nucleic acid sequences that encodes for a trehalose transport protein and returning said genetically engineered immune cells to the subject.

24. (canceled)

25. (canceled)

26. A kit comprising a pharmaceutical composition of claim 14 and a pharmaceutical composition comprising trehalose in pharmaceutical grade purity and a pharmaceutically acceptable carrier.

27. A method of determining suitability of a subject suffering from a solid cancer to be treated by a method of claim 18, the method comprising measuring glucose concentration in a TME of said solid cancer, wherein a glucose concentration below a predetermined threshold indicates said subject is suitable for treatment and wherein said predetermined threshold is the glucose concentration in the organ in which said solid cancer is found in a control subject or a non-cancerous location in said organ in said subject, and performing a method of claim 18 on a suitable subject thereby determining suitability of a subject.

28. (canceled)

29. A method of producing a cell of claim 1, the method comprising:
a. providing a mammalian immune cell; and
b. introducing into said provided mammalian immune cell a nucleic acid sequence encoding for a trehalase enzyme and a nucleic acid sequence encoding a trehalose transporter protein to produce an engineered cell;
thereby producing a cell of claim 1.

30. The method of claim 29, further comprising testing that said engineered cell is capable of surviving in the presence of trehalose and the absence or depletion of glucose.
